(19) United States
(12) Patent Application Publication
(10) Pub. No.: US 2010/0297230 A1
(43) Pub. Date: Nov. 25, 2010

(54) COMPOSITIONS AND METHODS FOR PROSTATE AND MENSTRUAL/MENOPAUSAL RELATED HEALTH

(75) Inventor: RONALD S. FLETCHER, Oakville (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: KEY NATURALS INC., Oakville (CA)

(21) Appl. No.: 12/784,180

(22) Filed: May 20, 2010

Related U.S. Application Data
(60) Provisional application No. 61/180,306, filed on May 21, 2009.

(30) Foreign Application Priority Data
May 21, 2009 (CA) 2666446

(51) Int. Cl. A61K 36/31; A61K 9/36; A61P 35/00; A61P 15/00; A61P 13/00; A61P 5/24

(52) U.S. Cl. 424/479; 424/755

(57) ABSTRACT

The disclosure provides compositions and methods and maintaining prostate health and relieving menstrual and menopausal related symptoms. The compositions comprise at least two of an icariin component, a quercetin component, a Maca component, a cocoa polyphenols component, and/or a rosmarinic acid component.

### RELATED APPLICATION

This application claims the benefit of 35 USC 119 based on the priority of co-pending U.S. Provisional Patent Application 61/180,306, filed May 21, 2009 and on co-pending CA patent application 2,666,446, filed May 21, 2009, each of those applications being incorporated herein in their entirety by reference.

### FIELD

The disclosure relates to methods, uses and compositions for maintaining urological, hormonal and reproductive health and particularly to methods and compositions for maintaining healthy prostate function and relieving menstrual related and menopausal related symptoms in a subject.

### INTRODUCTION

Icariin has been studied for its effects on prostate. Icaritin, a breakdown product of icariin, strongly inhibited growth of advanced human prostate carcinoma PC-3 cells. No risks were reported (Huang, X., Danyan, Z., Yijia, L., (2007) Eur. J. Pharm. 564: 26-36).

Icariin has also been studied for its effects on post menopausal bone loss. A randomized double blind placebo controlled trial to determine the effects of daily dose of Epimedium derived flavonoids (60 mg of icariin, 15 mg daidzein and 3 mg genistein) on preventing bone loss in late postmenopausal women found daily dosage of Epimedium derived flavonoids over 25 months increased bone mass density compared to placebo which decreased from baseline. No detectable hyperplasia was found on the uterus. No negative effects were reported during 24 months of administering Epimedium flavonoids (Zhang et al., (2007) Journal of Bone and Mineral Research 22: 1072-1079).

A study reported the serum levels of icariin achievable from a single dose of Epimedium tea (50 g leaves per 500 mL). The total dose of icariin ingested was 12.6 mg. The peak serum icariin concentrations reached from ingesting the tea were 2.1 ng/mL. No risks were reported (Gong et al. (2007) Journal Chromatography B 860: 166-172). A second study was designed to determine the serum levels of icaritin and desmethylicaritin from a single dose of Epimedium tea. The total dose of icariin was 12.6 mg, icaritin was 0.75 mg and desmethyl-icaritin was 0.19 mg ((50 g leaves per 500 mL). Icaritin levels peaked at 8 hours ranging from 0.61 nM to 5.11 nM in subjects, indicating a conversion of icariin to icaritin in vivo. Desmethylicaritin was not detected. No risks were reported (Shen et al. (2007) Journal Chromatography B 857: 47-52.).

A study on icaritin and its effects on benign prostatic hyperplasia (BPH) revealed that icaritin reduces the growth of human prostatic smooth muscle cells and induces apoptosis. The mode of action is independent of estrogen receptor signaling and decreases cyclin D1 and CDK4 expression. (Chen et al., 2010 Amino Acids 38:1505-1513).

Maca has also been studied for its effects on the prostate and hormone levels. Rats treated with testosterone enanthate to induce prostatic hyperplasia were treated with either red maca containing 0.1 mg benzylglucosinolate or 0.1 mg finasteride and compared to a control group to determine whether the treatments would reduce prostate weight in rats with prostatic hyperplasia. A dose dependent reduction in prostate weight was observed with an increase of the dose of benzylglucosinolate in red maca. Benzylglucosinolate at 0.1 mg was as effective at reducing prostate weight as 0.1 mg finasteride. Red maca did not reduce seminal vesicle weight compared to finasteride. No risks were reported. (Circero, (2010) Recent Progress in Medicinal Plants 27:347-362; Gonzales et al., (2008) ANDROLOGIA 40:179-185; Gonzales et al., (2007) Asian Journal of Andrology 9: 245-251).

In another study rats were treated either with 3 different types of maca extracts to determine the effect on ventral prostate and seminal vesicle size in healthy specimens or were injected with testosterone enanthate and treated with Red Maca to observe effects on prostate and seminal vesicle weight, and serum testosterone and estradiol levels. Red Maca in conjunction with testosterone enanthate treatments significantly reduced ventral weight, and had no effect on the testes, seminal vesicles, kidneys, spleen, liver, lungs, and heart. At 42 days after testosterone enanthate injection and Red Maca treatment, prostate size was the same as that of the controls (Gonzales et al., (2005) Reproductive Biology and Endocrinology 3: 5-20). No risks were reported.

A randomized double blind placebo controlled parallel trial to determine the effects of daily dose of 2 levels of Maca on serum levels of testosterone, estradiol, progesterone, follicle stimulating hormone, luteinizing hormone and prolactin. Men were placed in 6 groups based on dosage and type of administration (tablet vs. gelcap). When compared with placebo, Maca had no effect on any of the serum reproductive hormone concentrations over time. No risks were reported (Gonzales et al., (2003) Journal of Endocrinology 176: 163-168).

A previous randomized double blind placebo controlled parallel trial to determine the effects of daily dose of 2 levels of Maca on serum levels of testosterone and estradiol, their effects on sexual desire and an assessment of depression and anxiety using Hamilton Rating scales was conducted. When compared with placebo, Maca had no effect on serum testosterone or estradiol levels. No changes were observed in the Hamilton scores for anxiety or depression. Logistic regression did show an improvement in sexual desire at 8 and 12 weeks of treatment with Maca (Gonzales et al. (2002) Andrologia 34: 367-372). No risks were reported.

Quercetin has also been studied for its effect on the prostate. Rats (120) were divided into 4 groups and injected with 4 treatments of quercetin (0, 30, 90, 270 mg/kg) daily. At the end of the study, the rats were assessed for the effect of quercetin treatment on prostate weight, testes, seminal vesicles, sperm quality and vas deferens. Prostate and seminal vesicle size decreased significantly (P<0.05) in the 270 mg/kg group compared to all other treatments. This group also had a positive effect on sperm quality and sexual organ function compared to all groups. No risks were reported (Taepongsorat et al., (2008) Asian Journal of Andrology 10: 249-258).

A case control study of diet and prostate cancer in western New York involving 433 prostate patients and 538 control patients observed a reduction in the risk of prostate cancer with the consumption of quercetin, and identified no risks (McCann et al., (2005) Nutrition and Cancer 53: 33-41).

A 7 day clinical trial of male and female subjects (18-55 yrs) to examine the pharmacokinetics of quercetin and determine the metabolites produced found that peak plasma concentrations were reached at 3.5 hours at a concentration of 463 ng/mL, the highest recorded levels of any study to date. Primary metabolites were methyl quercetin, glucuronide and sulfate conjugates. Some enterohepatic recycling was observed. No risks were reported (Moon et al., (2008) Biopharmaceutics and Drug Disposition 29: 205-217).

Quercetin has been studied for its effect on matrix metalloproteinase expression. Vijayababu et al. assessed the ability of quercetin to decrease the expression and activity of two matrix metalloproteinases, MMP2 and MMP9, key proteins in tumour invasion and metastasis. Quercetin inhibited both MM2 and MMP9 expression in a dose dependent manner and significantly inhibited both MMP2 and MMP9 activity in PC-3 cells (P<0.05) (Vijayababu et al. (2006) Molecular and Cellular Biology 287: 109:116).

Cocoa polyphenols have been studied for their effect on survival in animal prostate cancer models. In a study, rats were randomized into 4 groups, control plus 3 chemically induced by N-methylnitrosourea, in two phases, initiation and constant induction. Two of the 3 groups were given either 24 or 48 mg/kg of a preparation of 35% cocoa polyphenols to determine the effect of cocoa polyphenols on the survival rate of rats with prostate cancer. Tumour induced rats treated with cocoa at 24 mg/kg survived significantly longer than non-treated induced rats and cocoa treated at 48 mg/kg. No significant difference was found between survival of control group and 24 mg/kg cocoa treated. Higher amounts of cocoa at 48 mg/kg had a negative effect on survival compared to the 24 mg/kg treated group (Garcia-Ca~nas V et al., (2010) J. Pharm. Biomed. Anal. 51:290-304; Bisson, J F, Guardia-Llorens, M A, Hidalgo, S., Rozan, P., & M. Messaoudi. (2008) Eur. J. Cancer Prevention 17:54-61).

In another study rats were randomized into 4 groups, control plus 3 groups induced prostatic hyperplasia with subcutaneous injection of testosterone propionate. Two of the 3 groups were given either 24 or 48 mg/kg of a preparation of 35% cocoa polyphenols to determine the effect on prostate weight. Prostate weight was significantly reduced compared to the positive control (induced+placebo). No risks were reported (Bisson et al., (2007) Journal of Medicinal Food 10: 622-627). Bisson et al. reported another study also in the Journal of Medicinal Food. Rats were randomized into 4 groups, control plus 3 groups induced prostatic hyperplasia with subcutaneous injection of testosterone propionate. Two of the 3 groups were given either 24 or 48 mg/kg of a preparation of 35% cocoa polyphenols to assess the therapeutic potential of cocoa polyphenols in the treatment of established prostatic hyperplasia. Prostate weight and dihydrotesterone levels were significantly reduced in both cocoa polyphenol treatment levels compared to the positive control (induced+placebo). No risks were reported (Bisson et al., (2007) Journal of Medicinal Food 10: 628-635).

Cocoa polyphenols have been studied for effects on blood pressure. A randomized, controlled, investigator blinded, parallel group trial, 44 adults, aged 56-73 (24 men, 20 women) with hypertension had changes in blood pressure, nitric oxide and oxidative stress measured after short term cocoa polyphenol daily intake. Low habitual cocoa polyphenol intake reduced hypertension prevalence from 86% to 68% and effectively reduced blood pressure. No changes were observed in oxidative stress and vasodilative nitric oxide levels increased significantly (p<0.001). No risks were reported (Taubert et al., (2007) Journal of the American Medical Association 298: 49-60).

### SUMMARY

In an aspect, the disclosure provides a composition comprising two or more of an icariin component, a Maca component, a quercetin component and a cocoa polyphenols component.

In an embodiment, the composition comprises two or more of an icariin component, a Maca component, a quercetin component, a cocoa polyphenols component and a rosmarinic acid component.

In another embodiment the icariin component comprises an extract comprising at least 5% icariin active ingredient for example icariin or icaritin. In a further embodiment, the icariin component comprises an extract of an Epimedium species, such as Vancouveria hexandra, and/or Ranzania japonica. In a further embodiment, the amount of the icariin active ingredient is at least 5 mg, at least 15 mg, at least 25 mg, at least 50 mg, at least 65 mg, at least 80 mg, at least 100 mg, at least 150 mg, at least 200 mg, at least 300 mg, at least 400 mg, at least 500 mg, at least 600 mg, at least 700 mg, at least 800 mg, at least 900 mg, or at least 1000 mg.

In another embodiment, the quercetin component comprises an extract comprising at least 5% quercetin component. In yet a further embodiment, the amount of quercetin active ingredient is at least 20 mg, at least 40 mg, at least 50 mg, at least 65 mg, at least 80 mg, at least 95 mg, at least 100 mg, at least 150 mg, at least 200 mg, at least 300 mg, at least 400 mg, at least 500 mg, at least 600 mg, or at least 700 mg.

In another embodiment, the cocoa polyphenols component is an extract comprising at least 5% cocoa polyphenols component.

In an embodiment, the extract is an extract of Theobroma cacao.

In another embodiment, the amount of cocoa polyphenols active ingredient is at least 5 mg, at least 15 mg, at least 25 mg, at least 35 mg, at least 50 mg, at least 65 mg, at least 80 mg, at least 100 mg, at least 150 mg, or at least 200 mg.

In another embodiment, the Maca component comprises Maca root powder. In another embodiment, the Maca component comprises an extract of Maca root such as total Maca root or Maca hypocotyls. In a further embodiment, the Maca component comprises Maca root powder with a P.E. 4:1, P.E. 6:1, P.E. 10:1, P.E. 15:1 or S.E of about 0.2% to about 10.0% glucosinolates and/or macamides.

In an embodiment, the amount of Maca component comprises at least 5 mg, at least 15 mg, at least 25 mg, at least 50 mg, at least 65 mg, at least 80 mg, at least 100 mg, at least 150 mg, at least 200 mg, at least 300 mg, at least 400 mg, at least 500 mg, at least 600 mg, at least 700 mg, at least 800 mg, at least 900 mg, or at least 1000 mg.

In another embodiment, the rosmarinic acid component comprises an extract comprising at least 2% rosmarinic acid component. In another embodiment, the extract comprises an extract of Rosmarinus officinalis, Mentha spicata, Origanum sp, Thymus sp. Prunella sp. Melissa, Salvia, Lamiaceae herb and/or Perilla and/or combinations thereof.

In a further embodiment, the amount of rosmarinic acid active ingredient is at least 20 mg, at least 25 mg, at least 50 mg, at least 65 mg, at least 80 mg, at least 100 mg, at least 150 mg, at least 200 mg, at least 300 mg, at least 400 mg, or at least 500 mg.

In certain embodiments, at least one component is a powder. In an embodiment, the composition further comprising an excipient. In an embodiment the excipient is magnesium stearate

In certain embodiments, the composition is formulated as an oral dosage form. In an embodiment, the composition is in a solid dosage form. For example, in an embodiment, the composition is a powder suitable for use as a reconstituted liquid drink. In another embodiment the composition is in a liquid dosage form.

Another aspect provides a method for maintaining prostate health comprising administering to a subject in need thereof a composition of the disclosure.

In an embodiment, the composition is administered for prophylaxis to inhibit prostate cancer or prostatic hyperplasia and/or to maintain sexual function.

In another embodiment, the subject has prostate cancer or prostatic hyperplasia.

Another aspect provides a method of treating prostate cancer and/or prostatic hyperplasia comprising administering to a subject with prostate cancer or prostatic hyperplasia a composition of the disclosure.

A further aspect provides a method of relieving or treating menstrual related symptoms comprising administering to a subject in need thereof a composition of the disclosure, comprising two or more of an icariin component, a quercetin component, a Maca component, a cocoa polyphenols component and a rosmarinic acid component.

Further, as aspect provides a method of relieving or treating premenstrual syndrome comprising administering to a subject in need thereof, a composition of the disclosure, comprising two or more of an icariin component, a quercetin component, a Maca component, a cocoa polyphenols component and a rosmarinic acid component

Another aspect provides a method of relieving or treating menopausal related symptoms comprising administering to a subject in need thereof a composition of the disclosure, comprising two or more of an icariin component, a quercetin component, a Maca component, a cocoa polyphenols component and a rosmarinic acid component.

In an embodiment, the subject is administered a recommended dosage.

In another embodiment, the recommended dosage is about 1 to about 3 or more dosage units, for example capsules per day. In a further embodiment, the recommended dosage is about 1 to about 3 dosage units, for example capsules about every 4, about every 6, or about every 8 hours as needed.

A further aspect provides use of a composition of the disclosure for maintaining prostate health. In an embodiment, the composition is used prophylactically to inhibit prostate cancer or prostatic hyperplasia and/or to maintain sexual function.

Also provided in an aspect is use of a composition of the disclsoure for treating prostate cancer and/or prostatic hyperplasia, for relieving or treating menstrual related symptoms, for relieving or treating premenstrual syndrome and/or for relieving or treating menopausal related symptoms.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### DESCRIPTION OF VARIOUS EMBODIMENTS

### I. Definitions

The term “icariin component” as used herein refers to an ingredient comprising or optionally consisting of, an icariin active ingredient. The term “icariin active ingredient” as used herein means icariin (5-hydroxy-2-(4-methoxyphenyl)-8-(3-methylbut-2-enyl)-7-[(2S,3R,4S,5S,6R)-3,4,5-trihydroxy-6-(hydroxymethyl)oxan-2-yl]oxy-3-[(2S,3R,4R,5R,6S)-3,4,5-trihydroxy-6-methyloxan-2-yl]oxychromen-4-one), a metabolite of icariin, such as icaritin, or an analog of icariin, and/or a pharmaceutically acceptable salt, prodrug and/or solvate thereof, as well as mixtures thereof. The chemical structure of icariin is:

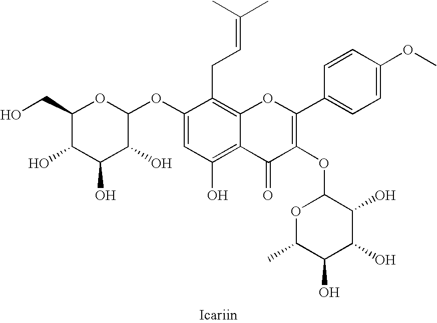

Icariin is a flavanol glycoside, typically isolated from an Epimedium plant. Accordingly, the icariin component can comprise, for example a plant or portion thereof (e.g leaves) comprising an icariin active ingredient such as icariin, for example comprising at least 5% icariin. For example, the icariin component can comprise a plant such as an Epimedium plant or portion thereof comprising an icariin active ingredient, including for example powdered, dried, crushed or ground plant (e.g. Epimedium powder comprising 40% icariin), leaves or the like; or an extract (i.e. the icariin component can be comprised in an extract), such as a plant extract for example an extract of an Epimedium species comprising an icariin active ingredient, for example comprising at least 5% icariin. The extract can be for example an extract of the leaves of an Epimedium plant.

The icariin component can be a powder, solution, or mixture and can for example be natural and/or synthetic. Icariin active ingredient derived from Epimedium is for example available commercially for example from Stryka Botanics, Hillsborough, N.J., USA.

The term “quercetin component” as used herein refers to an ingredient comprising, or optionally consisting of, a quercetin active ingredient. The term “quercetin active ingredient” as used herein means quercetin (2-(3,4-dihydroxyphenyl)-3,5,7-trihydroxy-4H-chromen-4-one)), a metabolite of quercetin, for example methyl quercetin and glucuronide and sulfate conjugates of quercetin, and analogs of quercetin, and/or a pharmaceutically acceptable salt, prodrug and/or solvate thereof, as well as mixtures thereof. The chemical structure of quercetin is:

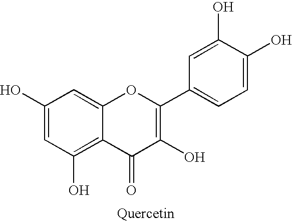

Quercetin is a plant-derived flavonoid. The quercetin component can comprise, for example a plant or a portion thereof (e.g. leaves) comprising a quercetin active ingredient, for example comprising at least 5% quercetin, such as the leaves of Saphora japonica or portions thereof, including for example dried, crushed or ground plant, leaves or the like; or an extract (i.e. the quercetin component can be comprised in an extract), such as a plant extract, for example an extract of Saphora japonica which comprises a quercetin active ingredient, for example comprising at least 5% quercetin. The extract can be an extract of the whole Saphora japonica plant, or a part such leaves. The quercetin component can be a powder solution, or mixture and can be natural, semi-synthetic or synthetic and/or mixtures thereof.

The term “rosmarinic acid component” as used herein refers to an ingredient comprising or optionally consisting of a rosmarinic acid active ingredient. The term “rosmarinic acid active ingredient” as used herein means rosmarinic acid ((2R)-2-[[(2E)-3-(3,4-Dihydroxyphenyl)-1-oxo-2-propenyl]]oxy]-3-(3,4-dihydroxyphenyl)propanoic acid), and/or or a pharmaceutically acceptable salt, prodrug and/or solvate thereof, as well as mixtures thereof. The chemical structure of rosmarinic acid is:

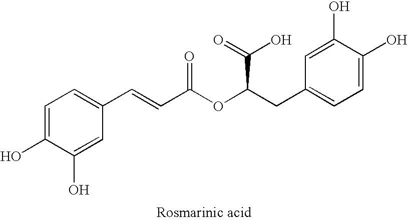

Rosmarinic acid is a naturally occurring (e.g. plant derived) polyphenol. The rosmarinic acid component can be for example a plant or a portion thereof (e.g. leaves) comprising a rosmarinic acid active ingredient, for example comprising at least 2% rosmarinic acid, for example plants or portions of Rosmarinus officinalis, Mentha spicata, Origanum sp, Thymus sp. Prunella sp. Melissa, Salvia or Perilla plants and/or combinations thereof, including for example dried, crushed and/or ground plant, leaves or the like; or an extract (i.e. the rosmarinic acid component can be comprised in an extract), such as a plant extract, for example an extract of Rosmarinus officinalis, Mentha spicata, Origanum sp, Thymus sp. Prunella sp. Melissa, Salvia, or Perilla and/or combinations thereof, comprising for example, at least 2% rosmarinic acid active ingredient. Additional plant species are known to comprise rosmarinic acid and are also suitable for use with the compositions and methods of the disclosure. The extract can be an extract of the whole plant, or a part such as the leaves. The rosmarinic acid component can be a powder, solution, or mixture and can be natural.

The term “cocoa polyphenol component” as used herein refers to an ingredient comprising or optionally consisting of a cocoa polyphenol active ingredient. The term “cocoa polyphenol active ingredient” as used herein means polyphenols from cocoa and/or pharmaceutically acceptable salts, prodrugs and/or solvates thereof, as well as mixtures thereof. The cocoa polyphenols component can be for example, a cocoa plant or a portion thereof (e.g. seeds or beans) comprising a polyphenol active ingredient, such as a Theobroma cacao plant or portion thereof, including for example dried, crushed and/or ground plant and/or seeds or the like; or an extract (e.g. the cocoa polyphenols component can be comprised in an extract, including a purified extract or standardized extract), such as a plant extract, for example an extract of Theobroma cacao seeds which comprises cocoa polyphenols. The extract can be an extract of the whole plant, or a part such as the seeds. The cocoa polyphenols component can be a powder, solution, or mixture and can be natural, semi-synthetic or synthetic and/or mixtures thereof.

The term “Maca component” as used herein refers to an ingredient comprising or optionally consisting of Maca (Lepidium meyenii) for example root or hypocotyls comprising a Maca active ingredient. The term “Maca active ingredient as used herein means glucosinates and/or macamides, for example at least 0.2% glucosinolates and/or macamides and/or from about 0.2% to about 5% glucosinolates and/or macamides. The Maca component can comprise for example Maca plant or a portion thereof (e.g. root and/or hypocotyls), including for example dried, crushed and/or ground Maca root and/or Maca hypocotyls including for example total Maca root powder; or an extract of Maca root or Maca hypocotyl, having for example an extract to powder ratio (PE), of for example P.E. 4:1, P.E. 6:1, P.E. 10:1 or P.E. 15:1, and/or comprises a select Standardized Extract (S.E) extract, with for example, about 0.2-5.0% glucosinolates, such as benzylglucosinolate and/or allyl glucosinolate, or macamides. An extract to powder ratio of 4:1 means that the extract is a 4X concentrate of the source material. A standardized extract refers to an extract comprising the specified quantity of the standardized ingredient.

The term “glucosinolates” as used herein refers to a class of organic compounds that contain sulfur and nitrogen and are derived from glucose and an amino acid as well as pharmaceutically acceptable salts, prodrugs or solvates and/or mixtures thereof. There are about 120 glucosinolates known to occur in plants and these include, for example benzyl glucosinolate.

The term “macamides” as used herein refers to a class of secondary metabolites found in Maca (Lepidium meyenii) as well as pharmaceutically acceptable salts, prodrugs or solvates and/or mixtures thereof. The main macamides include for example N-benzylhexadecanamide, N-benzyl-(9Z)-octadecenamide, N-benzyl-(9Z,12Z)-octadecadienamide, N-benzyl-(9Z,12Z,15Z)-octadecatrienamide and N-benzyloctadecanamide (McCollom, M M, Villinski J R, McPhail, K L, Craker, L E, Gafner, S. (2005) Phytochemical Anal 16:463-469).

The term “mixture” as used herein, means a composition comprising two or more of ingredients. In an embodiment a mixture is a mixture of two or more distinct ingredients. In a further embodiment, when a compound is referred to as a “mixture”, this means that it can comprise two or more “forms” of the compound, such as, salts, solvates, prodrugs or, where applicable, stereoisomers of the compound in any ratio. A person of skill in the art would understand that an ingredient in a mixture can also exist as a mixture of forms. For example, a compound may exist as a hydrate of a salt or as a hydrate of a salt of a prodrug of the compound. All forms of the compounds disclosed herein, in any ratio or combination, are within the scope of the present disclosure.

The term “component of the disclosure” as used herein refers to an icariin component, Maca component, quercitin component, cocoa polyphenols component and/or rosmarinic acid component; and/or pharmaceutically acceptable salts, solvates and/or prodrugs as well as mixtures thereof; and similarly “components of the disclosure” refers to two or more of such components.

The term “composition of the disclosure” as used herein refers to a composition comprising two or more of an icariin component, Maca component, quercitin component, cocoa polyphenols component and rosmarinic acid component; and/or pharmaceutically acceptable salts, solvates and/or prodrugs thereof.

The term “salt of or “salts thereof' as used herein refers to any pharmaceutically acceptable salt.

The term “pharmaceutically acceptable” means compatible with the treatment of animals, in particular, humans.

The term “pharmaceutically acceptable salt” means a component described herein paired with a anion or a cation of a pharmaceutically acceptable inorganic or organic acid or pharmaceutically acceptable inorganic or organic base which is suitable for, or compatible with, the treatment of subjects, in particular human subjects. Suitable anions include, but are not limited to, halides (iodide, chloride, bromide or fluoride), sulfates, formates, acetates, alkylsulfonates, aryl sulfonates and phosphates. Suitably the anion is a halide. Suitable cations include for example alkali metals such as Na⁺ and K⁺ or ammonium cations.

The term “maintaining prostate health” as used herein refers to reducing or inhibiting prostate cell grow and/or enlargement of the prostate gland, and/or supporting/maintaining normal function of the prostate. For example, in men over the age of 40 years, the prostate begins to enlarge (benign prostatic hyperplasia) and continues to grow with age, other prostate diseases may develop, such as prostate cancer and/or prostitis. These conditions can cause symptoms such as increased frequency and urgency to urinate, incontinence (loss of urinary control), difficulty starting/stopping urination, weak urine stream, bladder distention/pressure and painful and/or bloody urination. Maintaining/supporting normal prostate health and function refers to reducing or inhibiting excessive prostate cell growth (e.g. any one or more changes) and/or reducing or inhibiting symptoms associated with prostate disease (e.g. any one or more symptoms). In addition, since the prostate gland is part of the reproductive and urological system maintaining a healthy prostate function also includes maintaining healthy urinary and sexual function.

The term “sexual dysfunction” as used herein refers to a difficulty experienced by an individual during a sexual activity, including desire, arousal or orgasm, and includes without limitation, erectile dysfunction. The term “menstrual related symptoms” as used herein refers to menstrual symptoms, premenstrual symptoms, and/or symptoms associated with premenstrual syndrome.

The term “menstrual symptoms” as used herein refers to symptoms that are associated with menstruation. For example, menstrual symptoms include for example abdominal pain, back pain, cramps, breast tenderness, mood changes, headaches, nausea and/or dizziness.

The term “premenstrual syndrome” or PMS as used herein refers to a collection of symptoms linked to the menstrual cycle and include for example depression, anger, irritability, anxiety, bloating, weight gain, abdominal pain, muscles and/or joint pain, breast pain and swelling, headache, fatigue, difficulty sleeping, difficulty concentrating, confusion, food cravings, lack of sexual interest and/or decreased efficiency.

The term “menopausal related symptoms” as used herein refers to menopausal, pre-menopausal including PMS, post-menopausal, and peri-postmenopausal symptoms. These symptoms include for example hot flashes, night sweats, sleep disturbances, vaginal dryness, fatigue, weight gain, urinary tract infections, loss of bone mass, irritability, mood changes.

As used herein, the phrase “effective amount” or “therapeutically effective amount” means an amount effective, at dosages and for periods of time necessary to achieve the desired result. For example in the context or treating menopausal related symptoms, an effective amount is an amount that for example reduces or alleviates a symptom associated with menopause such as sleeplessness, and hot flashes compared to the response obtained without administration of the compound. Effective amounts may vary according to factors such as the disease state, age, sex, and/or weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

In general, prodrugs will be functional derivatives which are readily convertible in vivo into the compound from which it is notionally derived. Prodrugs may be conventional esters formed with available hydroxy and/or amino groups. For example, an available OH and/or NH₂ may be acylated using an activated acid in the presence of a base, and optionally, in inert solvent (e.g. an acid chloride in pyridine). Some common esters which have been utilized as prodrugs are phenyl esters, aliphatic (C₈-0₂₄) esters, acyloxymethyl esters, carbamates and amino acid esters. In certain instances, prodrugs are those in which hydroxy and/or amino groups in a compound are masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The term “solvate” as used herein means a compound, or a pharmaceutically acceptable salt or prodrug of such a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the identity of the compound and the solvate. In general, solvates are formed by dissolving the compound in the appropriate solvent and isolating the solvate by cooling or using an antisolvent. The solvate is typically dried or azeotroped under ambient conditions.

The term “subject” as used herein includes all members of the animal kingdom including mammals, and suitably refers to humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with prostate cancer can be treated with a composition of the disclosure, to inhibit progression or alternatively a subject in remission can be treated with a composition described herein to prevent recurrence. Treatment methods comprise administering to a subject a therapeutically effective amount of a compound described herein and optionally consists of a single administration, or alternatively comprises a series of applications. For example, the compositions described herein are administered at least once a week. However, in another embodiment, the compositions are administered to the subject from about one time per week to at least about once daily for a given treatment. In another embodiment, the composition is administered at least twice daily. The length of the treatment period depends on a variety of factors, such as the severity of the disease, the age of the patient, the concentration, the activity of the components described herein, and/or a combination thereof. It will also be appreciated that the effective dosage of the composition used for the treatment or prophylaxis may increase or decrease over the course of a particular treatment or prophylaxis regime. Changes in dosage may result and become apparent by standard diagnostic assays known in the art. In some instances, chronic administration may be required.

The term “relieving” as used herein refers reducing the intensity of one or more symptoms associated with a condition, for example reducing the intensity of pain associated prior to and/or during menstruation.

The term “cancer” as used herein means any of various types of neoplasms and/or malignancies characterized by abnormal or uncontrolled cell proliferation as compared to normal tissue.

The term “a cell” as used herein includes a plurality of cells. Administering a compound to a cell includes in vivo, ex vivo and in vitro treatment.

The term “solid dosage form” is to be understood to refer to individually coated tablets, capsules, including for example gelatin capsules, granules or other non-liquid dosage forms which are suitable, for example, for oral administration. It is to be understood that the solid dosage form includes, but is not limited to, non-controlled release, controlled release and time-controlled release dosage form units, employed preferably in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet.

The term “liquid dosage form” is to be understood to refer to non-solid dosage forms suitable for, but not limited to, oral, intravenous, subcutaneous, intramuscular, or intraperitoneal administration. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “at least”, “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### II. Compositions

Disclosed herein, in an aspect of the disclosure, are compositions comprising two or more of an icariin component, a Maca component, a quercetin component and a cocoa polyphenols component. These compositions are useful for maintaining urological, hormonal and reproductive health, particularly for maintaining prostate health.

Another aspect of the disclosure provides compositions comprising two or more of an icariin component, a Maca component, a quercetin component, a cocoa polyphenols component and a rosmarinic acid component. These compositions are useful for maintaining health, particularly for relieving menstrual related and menopausal related symptoms.

Compositions comprising two or more of an icariin component, a Maca component, a quercetin component, a cocoa polyphenols component and a rosmarinic acid component are herein referred to as compositions of the disclosure.

In an embodiment, the composition comprises an icariin component, a Maca component, a quercetin component, and a cocoa polyphenols component.

In another embodiment, the composition comprises an icariin component, a Maca component, a quercetin component, a cocoa polyphenols component and a rosmarinic component.

In a further embodiment, the composition comprises a Maca component, a quercetin component, and a cocoa polyphenols component.

The composition is optionally formulated suitably for oral administration, for example in unit dosage form such as a capsule. The composition is in an embodiment formulated suitably for use as a reconstituted liquid drink.

The combination of components can provide improved benefit over individual components. For example, with respect to a composition comprising icariin, quercetin, cocoa polyphenols, Maca and rosmarinic acid components, or a composition comprising quercetin, cocoa polyphenols, Maca and rosmarinic acid components, pain relief and mood enhancement are provided simultaneously thereby enhancing the combination's overall efficacy for relieving menstrual related symptoms such as menstrual related symptoms, such as premenstrual syndrome symptoms and/or menopausal related symptoms. Each of the components is described and examples of their medicinal properties are provided below.

Icariin Component Icariin is an isoprenylflavonoid compound and is along with the metabolite icaritin, found for example in and derivable from several species of plants in the Epimedium family, which are commonly known for example as Rowdy Lamb Herb, Barrenwort, Bishop's Hat, Fairy Wings, Horny Goat Weed or Yin Yang Huo. Over 60 cultivated species of Epimedium are known, many of which comprise icariin (Shen et al., (2007)). Examples of species comprising icariin and/or icaritin include Epimedium brevicornum, Epimedium grandiflorum and Epimedium sagittatum. Any extract comprising at least 5% icariin active ingredient, for example, any extract of any species of Epimedium comprising at least 5% icariin active ingredient is suitable for use in the compositions and methods described herein. Other species that contain icariin include Vancouveria hexandra, and Ranzania japonica.

The Systematic (IUPAC) name for icariin is 5-hydroxy-2-(4-methoxyphenyl)-8-(3-methylbut-2-enyl)-7-[(2S,3R,4S,5S,6R)-3,4,5-trihydroxy-6-(hydroxymethyl)oxan-2-yl]oxy-3-[(2S,3R,4R,5R,6S)-3,4,5-trihydroxy-6-methyloxan-2-yl]oxychromen-4-one. Accordingly, the icariin component comprises in an embodiment, 5-hydroxy-2-(4-methoxyphenyl)-8-(3-methylbut-2-enyl)-7-[(2S,3R,4S,5S,6R)-3,4,5-trihydroxy-6-(hydroxymethyl)oxan-2-yl]oxy-3-[(2S,3R,4R,5R,6S)-3,4,5-trihydroxy-6-methyloxan-2-yl]oxychromen-4-one. A metabolite of icariin is icaritin. Accordingly, in an embodiment, the icariin component comprises icaritin. Icaritin can be produced synthetically or semi-synthetically for example, from an icariin extract. For example it can be prepared by simple acid hydrolysis using acetic acid with heating to remove the sugars over a 24 hour period then extracting with ethyl acetate and drying in vacuo.

Further, pharmaceutically acceptable salts, prodrugs and/or solvates of icariin and/or icaritin as well as mixtures thereof can be used in the compositions and methods of the disclosure.

In an embodiment, the icariin component comprises a mixture of two or more icariin active ingredients, for example two or more of icariin, synthetic or semi-synthetic icariins, metabolites, and/or pharmaceutically acceptable salts, prodrugs, or solvates as well as mixtures thereof. In an embodiment, the icariin component comprises an extract or a mixture of extracts.

In an embodiment, the extract comprising icariin active ingredient is a purified extract.

Accordingly in an embodiment, the icariin component, comprises at least 5% icariin active ingredient. In another embodiment the icariin component comprises at least 10%, at least 20%, at least 30%, at least 40%, at least 50%, at least 60%, at least 70%, at least 80%, at least 90%, at least 95%, or at least 98% icariin active ingredient. In another embodiment, the icariin component comprises about, 5%, about 10%, about 20%, about 30%, about 40%, about 50%, about 60%, about 70%, about 80%, about 90%, about 95%, or about 98% icariin active ingredient. In another embodiment, the icariin component comprises about 5% to about 98% icariin active ingredient, about 10% to about 98% icariin active ingredient, about 20% to about 98% icariin active ingredient, about 30% to about 98% icariin active ingredient, about 40% to about 98% icariin active ingredient, about 50% to about 98% icariin active ingredient about 60% to about 98% icariin, or about 70% to about 98% icariin active ingredient.

In an embodiment, the extract comprises an Epimedium extract. In another embodiment, the extract comprises an extract of Epimedium brevicornum. In another embodiment, Epimedium extract comprises an extract of Epimedium grandiflorum. In a further embodiment, the Epimedium extract comprises an extract of Epimedium sagittatum.

In another embodiment, the extract comprises an extract of Vancouveria hexandra or Ranzania japonica.

In an embodiment, the amount of icariin active ingredient in the composition is at least 5 mg, at least 15 mg, at least 25 mg, at least 50 mg, at least 65 mg, at least 80 mg, at least 100 mg, at least 125 mg, at least 150 mg, at least 200 mg, at least 300 mg, at least 400 mg, at least 500 mg, at least 600 mg, at least 700 mg, at least 800 mg, at least 900 mg, or at least 1000 mg. In an embodiment, the amount of icariin active ingredient in the composition is about 5 mg, about 15 mg, about 25 mg, about 50 mg, about 65 mg, about 80 mg, about 100 mg, about 125 mg, about 150 mg, about 200 mg, about 300 mg, about 400 mg, about 500 mg, about 600 mg, about 700 mg, about 800 mg, about 900 mg, or about 1000 mg. In another embodiment, the amount of icariin active ingredient in the composition is about 5 mg to about 1000 mg, about 15 mg to about 1000 mg, about 25 mg to about 1000 mg, about 50 mg to about 1000 mg, about 65 mg to about 1000 mg, about 80 mg to about 1000 mg, about 100 mg to about 1000 mg, about 150 mg to about 1000 mg, about 200 mg, to about 1000 mg, about 300 mg to about 1000 mg, about 400 mg to about 1000 mg, about 500 mg to about 1000 mg or about 600 mg to about 1000 mg. In another embodiment, the amount of icariin active ingredient in the composition is about 5 mg to about 500 mg, about 15 mg to about 500 mg, about 25 mg to about 500 mg, about 50 mg to about 500 mg, about 65 mg to about 500 mg, about 80 mg to about 500 mg, about 100 mg to about 500 mg, about 150 mg to about 500 mg, about 200 mg to about 500 mg, about 300 mg to about 500 mg, or about 400 mg to about 500 mg.

The amount of icariin component necessary to provide a particular amount of icariin active ingredient can be readily calculated by a person skilled in the art having regard to the concentration of icariin active ingredient in a particular icariin component. A person skilled in the art would understand that to provide for example, 50 mg of icariin active ingredient in a composition, 125 mg of an icariin component (e.g. Epimedium icariin powder) comprising 40% icariin active ingredient is necessary. Where the concentration of icariin active ingredient in an icariin component is not known, it can be determined for example using analytical means and comparing to known standards.

For example, 500 mg of a 40% Epimedium icariin powder will provide 200 mg of icariin active ingredient. A person skilled in the art would readily understand that when using a less concentrated icariin component, an increased amount of icariin component is required, for example if using a 20% Epimedium icariin powder, a person skilled in the art would recognize that 1000 mg of Epimedium icariin powder is required to provide 200 mg of icariin active ingredient.

In an embodiment, the composition is formulated suitably as a capsule or tablet. In an embodiment, the composition is formulated suitably for reconstitution as a liquid drink.

As an example, wherein the composition is provided as a formulation suitable for use as a reconstituted liquid drink, the icariin component can comprise, an amount of icariin active ingredient described herein for example for reconstitution in about 200 mL, about 250 ml, about 300 mL, about 350 mL, or about 400 mL. In an embodiment, the icarriin component in the composition comprises, at least, 50 mg, at least 100 mg, at least 200 mg, at least 300 mg, at least 400 mg, at least 500 mg, at least 1000 mg, at least 1500 mg, at least 2000 mg or more of Epimedium icariin powder, optionally about 40% Epimedium icariin powder, suitable for a reconstituted liquid drink, for example for making about 250 mL of reconstituted liquid drink.

### Quercetin Component

Quercetin is a plant derived flavonoid found and/or derivable for example from a number of plants including citrus fruit, buckwheat and onion. Quercetin is also present in for example Saphora japonica (Japanese scholartree), kale, hot peppers, and rutabagas. Any ingredient such as a purified extract comprising at least 5% quercetin active ingredient is suitable for use in the compositions and methods described herein. Quercetin is primarily sold in pure form and can be purchased for example from Stryka Botanics, Hillsborough, N.J., USA. The Systematic (IUPAC) name for quercetin is 2-(3,4-dihydroxyphenyl)-3,5,7-trihydroxy-4H-chromen-4-one. In an embodiment, the quercetin component comprises 2-(3,4- dihydroxyphenyl)-3,5,7-trihydroxy-4H-chromen-4-one. Metabolites of quercetin include for example methyl quercetin, glucuronide and sulfate conjugates. Accordingly in an embodiment, the quercetin component comprises methyl quercetin, glucuronide and/or sulfate conjugates.

Further, pharmaceutically acceptable salts, prodrugs and/or solvates or quercetin and/or quercetin metabolites as well as mixtures thereof can be used in the compositions and methods of the disclosure.

In an embodiment, the quercetin component comprises a mixture of two or more quercetin active ingredients, for example two or more of quercetin, quercetin metabolites, and/or pharmaceutically acceptable salts, prodrugs, or solvates as well as mixtures thereof.

In an embodiment, the extract comprising quercetin component comprises a purified extract.

Accordingly in an embodiment, the quercetin component comprises at least 5% quercetin active ingredient. In another embodiment the quercetin component comprises at least 10%, at least 20%, at least 30%, at least 40%, at least 50%, at least 60%, at least 70%, at least 80%, at least 90%, at least 95%, or at least 98% quercetin active ingredient. In another embodiment, the quercetin component comprises about, 5%, about 10%, about 20%, about 30%, about 40%, about 50%, about 60%, about 70%, about 80%, about 90%, about 95%, or about 98% quercetin active ingredient. In another embodiment, the quercetin component comprises about 5% to about 98% quercetin active ingredient, about 10% to about 98% quercetin active ingredient, about 20% to about 98% quercetin active ingredient, about 30% to about 98% quercetin active ingredient, about 40% to about 98% quercetin active ingredient, about 50% to about 98% quercetin active ingredient, about 60% to about 98% quercetin active ingredient, or about 70% to about 98% quercetin active ingredient.

In an embodiment, the amount of quercetin active ingredient in the composition is at least 40 mg, at least 50 mg, at least 65 mg, at least 80 mg, at least 95 mg, at least 100 mg, at least 150 mg, at least 200 mg, at least 300 mg, at least 400 mg, at least 500 mg, at least 600 mg, or at least 700 mg. In an embodiment, the amount of quercetin active ingredient in the composition is about 20 mg, about 30 mg, about 40 mg, about 50 mg, about 65 mg, about 80 mg, about 95 mg, about 100 mg, about 150 mg, about 200 mg, about300 mg, about 400 mg, about 500 mg, about 600 mg, or about 700 mg. In another embodiment, the amount of quercetin active ingredient in the composition is about 40 mg to about 700 mg, about 50 mg to about 700 mg, about 65 mg to about 700 mg, about 80 mg to about 700 mg, about 100 mg to about 700 mg, about 150 mg to about 700 mg, about 200 mg to about 700 mg, about 300 mg to about 700 mg, about 400 mg to about 700 mg, about 500 mg to about 700 mg or about 600 mg to about 700 mg. In another embodiment, the amount of quercetin active ingredient in the composition is about 20 mg to about 350 mg, about 25 mg to about 350 mg, about 50 mg to about 350 mg, about 65 mg to about 350 mg, about 80 mg to about 350 mg, about 100 mg to about 350 mg, about 150 mg to about 350 mg, about 200 mg to about 350 mg, or about 300 mg to about 350 mg.

The amount of quercetin component necessary to provide a particular amount of quercetin active ingredient can be readily calculated by a person skilled in the art having regard to the concentration of quercetin active ingredient in a particular quercetin component. A person skilled in the art would understand that to provide for example, 90 mg of quercetin active ingredient in a composition, 100 mg of an quercetin component comprising 90% quercetin active ingredient is necessary. Where the concentration of quercetin active ingredient in an quercetin component is not known, it can be determined for example using analytical means and comparing to known standards.

In an embodiment, the composition is formulated suitably as a capsule or tablet. In an embodiment, the composition is formulated suitably for reconstitution as a liquid drink.

As an example, wherein the composition is provided as a formulation suitable for use as a reconstituted liquid drink, the quercetin component can comprise an amount of quercetin active ingredient described herein for example for reconstitution in about 200 mL, about 250 ml, about 300 mL, about 350 mL, or about 400 mL. In an embodiment, the quercetin component in the composition comprises at least 20 mg, at least 40 mg, at least 100 mg, at least 200 mg, at least 300 mg, at least 400 mg, at least 500 mg, at least 600 mg or at least 700 mg of for example pure quercetin powder suitable for reconstituted liquid drink, for example for making about 250 mL of reconstituted liquid drink.

### Cocoa Polyphenols

Cocoa polyphenols, optionally cocoa flavanoids, are mixtures of polyphenols derived from cocoa (or cacao) plants, for example Theobroma cacao (e.g. cocoa seeds commonly known as cocoa beans). Any extract comprising at least 5% cocoa polyphenol active ingredient, for example an extract of Theobroma cacao comprising at least 5% cocoa polyphenol active ingredient, is suitable for use in the compositions and methods described herein.

Pharmaceutically acceptable salts, prodrugs and/or solvates of cocoa polyphenols can also be used in the compositions and methods of the disclosure.

In an embodiment, the cocoa polyphenols component comprises a mixture of two or more cocoa polyphenols active ingredients, for example two or more of pharmaceutically acceptable salts, prodrugs, or solvates thereof. In an embodiment, the cocoa polyphenols component comprises an extract or a mixture of extracts. In an embodiment, the extract comprises a Theobroma cacao extract. In an embodiment, the extract is a purified extract.

Accordingly in an embodiment, the cocoa polyphenols component comprises at least 5% cocoa polyphenols active ingredient. In another embodiment the cocoa polyphenols component comprises at least 10%, at least 20%, at least 30%, at least 40%, at least 50%, at least 60%, at least 70%, or at least 80% cocoa polyphenols active ingredient. In another embodiment, the cocoa polyphenols component comprises about 10%, about 20%, about 30%, about 40%, about 50%, about 60%, about 70%, about 80% or about 90% cocoa polyphenols active ingredient. In an embodiment, the cocoa polyphenols component comprises about 40% coco polyphenols active ingredient. In another embodiment, the cocoa polyphenols component comprises about 5% to about 90% cocoa polyphenols active ingredient, about 10% to about 90% cocoa polyphenols active ingredient, about 20% to about 90% cocoa polyphenols active ingredient, about 30% to about 90% cocoa polyphenols active ingredient, about 40% to about 90% cocoa polyphenols active ingredient, about 50% to about 90% cocoa polyphenols active ingredient, about 60% to about 90% cocoa polyphenols active ingredient, or about 70% to about 80% cocoa polyphenols active ingredient.

In an embodiment, the amount of cocoa polyphenols active ingredient in the composition is at least 30 mg, at least 40 mg, at least 50 mg, at least 65 mg, at least 80 mg, at least 100 mg, at least 150 mg, or at least 200 mg. In an embodiment, the amount of cocoa polyphenols active ingredient in the composition is about 5 mg, about 15 mg, about 25 mg, about 35 mg, about 50 mg, about 65 mg, about 80 mg, about 100 mg, about 150 mg, or about 200 mg. In another embodiment, the amount of cocoa polyphenols active ingredient in the composition is about 5 mg to about 200 mg, about 15 mg to about 200 mg, about 25 mg to about 200 mg, about 35 to about 200 mg, about 50 mg to about 200 mg, about 65 mg to about 200 mg, about 80 mg to about 200 mg, about 100 mg to about 200 mg, or about 150 mg to about 200 mg. In another embodiment, the amount of cocoa polyphenols active ingredient in the composition is about 5 mg to about 100 mg, about 15 mg to about 100 mg, about 25 mg to about 100 mg, about 35 mg to about 100 mg, about 50 mg to about 100 mg, about 65 mg to about 100 mg, or about 80 mg to about 100 mg.

The amount of cocoa polyphenols component necessary to provide a particular amount of cocoa polyphenols active ingredient can be readily calculated by a person skilled in the art having regard to the concentration of cocoa polyphenols active ingredient in a particular cocoa polyphenols component. A person skilled in the art would understand that to provide for example, 50 mg of cocoa polyphenols active ingredient in a composition, 125 mg of a cocoa polyphenols component (e.g. Theobroma cacao extract) comprising 40% cocoa polyphenols active ingredient is necessary. Where the concentration of cocoa polyphenols active ingredient in a cocoa polyphenols component is not known, it can be determined for example using analytical means and comparing to known standards.

As an example, 125 mg of a 40% Theobroma cacao extract will provide 50 mg of cocoa polyphenols active ingredient. A person skilled in the art would readily understand that when using a less concentrated cocoa polyphenols component, an increased amount of cocoa polyphenols component is required, for example if using a 20% Theobroma cacao extract, a person skilled in the art would recognize that 250 mg of Theobroma cacao extract is required to provide 50 mg of cocoa polyphenols active ingredient.

In an embodiment, the composition is formulated suitably as a capsule or tablet. In an embodiment, the composition is formulated suitably for reconstitution as a liquid drink.

As an example, wherein the composition is provided as a formulation suitable for use as a liquid beverage such as a tea, the cocoa polyphenols component can comprise an amount of cocoa polyphenols active ingredient described herein, for example for reconstitution in about 200 mL, about 250 ml, about 300 mL, about 350 mL, or about 400 mL. In an embodiment, the cocoa polyphenols component comprises at least 40 mg, at least 65 mg, at least 100 mg, at least 150 mg, at least 200 mg or at least 1 gram, at least 2 grams, at least 3 grams, at least 4 grams, or at least 5 grams of cocoa powder suitable for reconstituted liquid drink, for example for making about 250 mL of reconstituted drink.

### Maca Component

Maca (Lepidium meyenii) root and/or hypocotyls and/or extracts thereof comprise glucosinolates, for example benzyl glucosinolate and/or macamides. Any ingredient comprising Maca plant part, for example root and/or hypocotyl, for example in powder extract form, including for example extracts thereof having Powdered Extract (P.E.) 4:1; P.E. 6:1, P.E. 10:1; P.E. 15:1; Standardized Extract (S.E.), having at least about 0.2% glucosinolate and/or macamide content, optionally having about 0.2 to about 5.0% glucosinolates or macamides and/or a combination thereof, is suitable for use in the compositions and methods described herein.

In an embodiment, the Maca component comprises Maca root or hypocotyls and/or extracts thereof. In an embodiment, the Maca active ingredient comprises a glucosinolate such as benzyl glucosinolate and/or a macamide.

Further, pharmaceutically acceptable salts, prodrugs and/or solvates as well as mixtures of glucosinolates and/or macamides can be used in the compositions and methods of the disclosure.

In an embodiment, the Maca component comprises two or more of glucosinolates and/or macamides, and/or pharmaceutically acceptable salts, prodrugs, or solvates thereof. In an embodiment, the Maca component comprises an extract or a mixture of extracts.

In an embodiment, the Maca component comprises a Maca root extract, for example total Maca root extract. In another embodiment, the Maca component comprises a hypocotyl extract. In a further embodiment, the Maca component comprises an extract comprising Maca root powder, for example total Maca root powder. In another embodiment, the Maca component comprises a Powdered Extract (P.E.). In an embodiment, the Maca component comprises a powder with P.E. 4:1, P.E. 6:1, P.E. 10:1, or P.E. 15:1. As an example of what is meant by P.E., P.E. 10:1 in the context of a Maca component refers to an extract of Maca component that has been concentrated 10 times, for example, a macamide normally present at a level of 0.06% in root or hypocotyl powder is concentrated to a level of 0.6%. In a further embodiment, the Maca component comprises a Standardized Extract (S.E.) As an example of what is meant by S.E., a S.E. of 0.6% macamide refers to an extract such as a powdered extract which has been analytically tested and confirmed to have 0.6% macamide content. In an embodiment, the extract is a purified extract.

In an embodiment, the Maca component comprises at least 0.2% glucosinolates and/or macamides. In another embodiment, the Maca component comprises at least 0.3%, at least 0.4%, at least 0.5%, at least 0.75%, at least 1%, at least 1.5%, at least 2%, at least 2.5%, at least 3%, at least 3.5%, at least 4%, at least 4.5%, at least 5.0%, at least 6%, at least 7%, at least 8%, at least 9%, or at least 10% or more glucosinolates and/or macamides. In another embodiment, the Maca component comprises about 0.2%, about 0.3%, about 0.4%, about 0.5%, about 0.75%, about 1%, about 1.5%, about 2%, about 2.5%, about 3%, about 3.5%, about 4%, about 4.5%, about 5.0%, about 6%, about %, about 8%, about 9%, or about 10% or more glucosinolates and/or macamides.

In an embodiment, the Maca comprises benzyl glucosinate active ingredient. In another embodiment, the glucosinolates comprise benzyl glucosinolate and/or allyl glucosinolate.

In an embodiment, the amount of maca component wherein the maca component is a powdered extract of P.E. 10:1, is about 100 mg to about 500 mg. The amount of glucosinolates and/or macamides in such an amount depending on the plant and extract concentration, is likely to be within the range of 0.6 mg and 25 mg.

In an embodiment, the amount of Maca active ingredient in the composition comprises at least 0.1 mg, at least 0.2 mg, at least 0.5 mg, at least 0.75 mg, at least 1 mg, at least 2 mg, at least 3 mg, at least 4 mg, at least 5 mg, at least about 7.5 mg, at least 10 mg, at least 15 mg, at least 20 mg, or at least 25 mg.

In an embodiment, the amount of Maca active ingredient in the composition comprises about 0.1 mg, about 0.2 mg, about 0.5 mg, about 0.75 mg, about 1 mg, about 2 mg, about 3 mg, about 4 mg, about 5 mg, about 7.5 mg, about 10 mg, about 15 mg, about 20 mg, or about 25 mg.

As the concentration of Maca active ingredient can vary between Maca plants, the concentration of an active ingredient such as benzyl glucosinolate can be determined, for example, by analytical means and comparing to a set of standards or a standard curve. Determining the level of a person skilled the art would be familiar with methods for determining concentration of a Maca active ingredient such as benzyl glucosinolate. For example, benzyl glucosinolate concentrations in Maca extract can be determined via HPLC analysis of Maca extracts using reverse phase chromatography and comparing the AUC with a benzyl glucosinolate standard curve. In an embodiment, the amount of Maca benzyl glucosinolate active ingredient in the composition comprises at least 0.1 mg, at least 0.2 mg, at least 0.5 mg, at least 0.75 mg, at least 1 mg, at least 2 mg, at least 3 mg, at least 4 mg, at least 5 mg, at least 7.5 mg, at least 10 mg, or at least 15 mg.

In an embodiment, the amount of Maca benzyl glucosinolate active ingredient in the composition comprises about 0.1 mg, about 0.2 mg, about 0.5 mg, about 0.75 mg, about 1 mg, about 2 mg, about 3 mg, about 4 mg, about 5 mg, about 7.5 mg, about 10 mg or about 15 mg.

In an embodiment, the amount of Maca component in the composition comprises at least 15 mg, at least 25 mg, at least 50 mg, at least 65 mg, at least 80 mg, at least 100 mg, at least 150 mg, 175 mg, at least 200 mg, at least 300 mg, at least 400 mg, at least 500 mg, at least 600 mg, at least 700 mg, at least 800 mg, at least 900 mg, or at least 1000 mg. In another embodiment, the about of Maca component in the composition is about 15 mg, about 25 mg, about 50 mg, about 65 mg, about 80 mg, about 100 mg, about 150 mg, about 200 mg, about 300 mg, about 400 mg, about 500 mg, about 600 mg, about 700 mg, about 800 mg, about 900 mg, or about 1000 mg.

In another embodiment, the amount of the Maca component in the composition is about 15 mg to about 1000 mg, about 25 mg to about 1000 mg, about 50 mg to about 1000 mg, about 65 mg to about 1000 mg, about 80 mg to about 1000 mg, about 100 mg to about 1000 mg, about 150 mg to about 1000 mg, about 200 mg, to about 1000 mg, about 300 mg to about 1000 mg, about 400 mg to about 1000 mg, about 500 mg to about 1000 mg or about 600 mg to about 1000 mg. In another embodiment, the amount of the Maca component in the composition is about 15 mg to about 500 mg, about 25 mg to about 500 mg, about 50 mg to about 500 mg, about 65 mg to about 500 mg, about 80 mg to about 500 mg, about 100 mg to about 500 mg, about 150 mg to about 500 mg, about 200 mg to about 500 mg, about 300 mg to about 500 mg, or about 400 mg to about 500 mg.

Maca component necessary to provide a particular amount of Maca active ingredient can be readily calculated by a person skilled in the art having regard to the concentration of Maca active ingredient in a particular Maca component. Where the concentration of Maca active ingredient in a Maca component is not known, it can be determined for example using analytical means and comparing to known standards.

In an embodiment, the composition is formulated suitably as a capsule or a tablet. In an embodiment, the composition is formulated suitably for reconstitution as a liquid drink.

As an example, wherein the composition is provided as a formulation suitable for use as a liquid drink such as a tea, the Maca component can comprise an amount of Maca active ingredient described herein, for example for reconstitution in about 200 mL, about 250 ml, about 300 mL, about 350 mL, or about 400 mL. The Maca component comprises in an embodiment, at least 50 mg, at least 70 mg, at least 100 mg, at least 125 mg, at least 150 mg, at least 175 mg, at least 200 mg, or for example at least 10 grams, at least 20 grams, at least 30 grams, at least 40 grams, or at least 50 grams of for example, Maca root powder suitable for reconstituted liquid drink, for example for making about 250 mL of reconstituted liquid drink.

Maca for example Maca P.E. 10:1 can be purchased for example from Stryka Botanics, Hillsborough, N.J. USA.

### Rosmarinic Acid Component

Rosmarinic acid is a natural polyphenol carboxylic acid, found for example in many Lamiaceae and Boraginaceae herbs as well as others such as Rosmarinus officinalis, Mentha spicata, Origanum sp, Thymus sp. Prunella sp. Melissa, Salvia, Borage and Perilla. Any ingredient comprising at least 2% rosmarinic acid active ingredient, for example an extract of Rosmarinus officinalis comprising at least 2% rosmarinic acid, is suitable for use in the compositions and methods described herein.

The Systematic (IUPAC) name for rosmarinic acid is (2R)-2-[[(2E)-3-(3,4-Dihydroxyphenyl)-1-oxo-2-propenyl]]oxy]-3-(3,4-dihydroxyphenyl)propanoic acid. Accordingly, in an embodiment, the rosmarinic acid component comprises (2R)-2-[[(2E)-3-(3,4-Dihydroxyphenyl)-1-oxo-2-propenyl]]oxy]-3-(3,4-dihydroxyphenyl)propanoic acid. Synthetic and semi-synthetic sources of rosmarinic acid active ingredient are suitable in the compositions and methods described herein. Accordingly, the rosmarinic acid component comprises in an embodiment, synthetic rosmarinic acid active ingredient or semi-synthetic rosmarinic acid active ingredient.

Further, pharmaceutically acceptable salts, prodrugs and/or solvates of rosmarinic acid can be used in the compositions and methods of the disclosure.

In a further embodiment, the rosmarinic acid component comprises a mixture of two or more of synthetic or semi-syntheticrosmarinic acid active ingredient, and/or pharmaceutically acceptable salts, prodrugs, or solvates thereof.

Accordingly in an embodiment, the rosmarinic acid component comprises at least 2% rosmarinic acid active ingredient. In another embodiment the rosmarinic acid component comprises at least 5%, at least 10%, at least 20%, at least 30%, at least 40%, at least 50%, at least 60%, at least 70%, or at least 80% rosmarinic acid active ingredient. In another embodiment, the rosmarinic acid component comprises about 2% rosmarinic acid active ingredient. In another embodiment the rosmarinic acid component comprises about 5%, about 10%, about 20%, about 30%, about 40%, about 50%, about 60%, about 70%, or about 80% rosmarinic acid active ingredient In another embodiment, the rosmarinic acid component comprises about 2% to about 80% rosmarinic acid active ingredient, about 5% to about 80% rosmarinic acid active ingredient, about 10% to about 80% rosmarinic acid active ingredient, about 20% to about 80% rosmarinic acid active ingredient, about 30% to about 80% rosmarinic acid active ingredient, about 40% to about 80% rosmarinic acid active ingredient, about 50% to about 80% rosmarinic acid active ingredient, about 60% to about 80% rosmarinic acid active ingredient, or about 70% to about 80% rosmarinic acid active ingredient.

In an embodiment, the rosmarinic acid component comprises an extract or a mixture of extracts.

In an embodiment, the rosamarinic acid component comprises an extract of Rosmarinus officinalis, Mentha spicata, Origanum sp, Thymus sp. Prunella sp. Melissa, Salvia, and/or Perilla and/or combinations thereof. In another embodiment, rosamarinic acid component comprises an extract of a Lamiaceae herb.

In an embodiment, the extract is a purified extract.

In an embodiment, the amount of rosmarinic acid active ingredient in the composition is at least 20 mg, at least 25 mg, at least 50 mg, at least 65 mg, at least 80 mg, at least 100 mg, at least 120 mg, at least 150 mg, at least 200 mg, at least 300 mg, at least 400 mg, at least 500 mg, at least 600 mg, at least 700 mg, at least 800 mg, at least 900 mg or at least 1000 mg. In another embodiment, the amount of the rosmarinic active ingredient in the composition comprises about 20 mg, about 25 mg, about 50 mg, about 65 mg, about 80 mg, about 100 mg, about 150 mg, about 200 mg, about 300 mg, about 400 mg, about 500 mg, about 600 mg, about 700 mg, about 800 mg, about 900 mg or about 1000 mg. In another embodiment, the amount of rosmarinic acid active ingredient in the composition is about 20 mg to about 500 mg, about 25 mg to about 500 mg, about 50 mg to about 500 mg, about 65 mg to about 500 mg, about 80 mg to about 500 mg, about 100 mg to about 500 mg, about 150 mg to about 500 mg, about 200 mg to about 500 mg, about 300 mg to about 500 mg, about 400 mg to about 500 mg. In another embodiment, the amount of rosmarinic acid ingredient in the composition is about 20 mg to about 250 mg, about 25 mg to about 250 mg, about 50 mg to about 250 mg, about 65 mg to about 250 mg, about 80 mg to about 250 mg, about 100 mg to about 250 mg, about 150 mg to about 250 mg, or about 200 mg to about 250 mg.

The amount of rosmarinic acid component necessary to provide a particular amount of rosmarinic acid active ingredient can be readily calculated by a person skilled in the art having regard to the concentration of rosmarinic acid active ingredient in a particular rosmarinic acid component. A person skilled in the art would understand that to provide for example, 50 mg of rosmarinic acid active ingredient in a composition, 125 mg of a rosmarinic acid component comprising 40% rosmarinic acid active ingredient is necessary. Where the concentration of rosmarinic acid active ingredient in a rosmarinic acid component is not known, it can be determined for example using analytical means and comparing to known standards.

As an example, 125 mg of a 40% extract will provide 50 mg of rosmarinic acid active ingredient. A person skilled in the art would readily understand that when using a less concentrated rosmarinic acid component, an increased amount of Rosmarinic acid component is required, for example if using a 20% extract, a person skilled in the art would recognize that 250 mg of extract is required to provide 50 mg of Rosmarinic acid active ingredient.

In an embodiment, the composition is formulated suitably as a capsule or tablet. In an embodiment, the composition is formulated suitably for reconstitution as a liquid drink.

As an example, wherein the composition is provided as a formulation suitable for use as a liquid drink such as a tea, the rosmarinic acid component can comprise an amount of rosmarinic acid active ingredient described herein, for example for reconstitution in about 200 mL, about 250 ml, about 300 mL, about 350 mL, or about 400 mL. In an embodiment, the rosmarinic acid component comprises at least 75 mg, at least 100 mg, at least 150 mg, at least 200 mg, at least 250 mg, at least 300 mg, at least 400 mg, at least 500 mg, at least 1 gram, at least 10 grams, at least 20 grams, at least 30 grams, at least 40 grams, at least 50 grams, of extracted herb leaves, such as Rosmarinus officinalis, suitable for reconstituted liquid drink, for example for making about 250 mL of reconstituted liquid drink.]

Rosmarinic acid extract can be purchased for example from PL Thomas Morristown, N.J. USA.

In an embodiment, the composition is a pharmaceutical composition.

In another embodiment, the composition is in dosage form, for example unit dosage form.

In a further embodiment, the pharmaceutical composition is formulated for oral administration or injection.

Components of the disclosure are available commercially, for example as described herein.

The compositions described herein can also be prepared by per se known methods for the preparation of pharmaceutically acceptable compositions that can be administered to subjects, such that an effective quantity of the active substance is combined in a mixture with a pharmaceutically acceptable vehicle and/or excipient (i.e. filler).

In an embodiment of the disclosure the pharmaceutical composition contains about 1% to about 10%, suitably about 10% to about 25%, of one or more components of the disclosure, for example by volume and/or weight. In an embodiment, a minimum amount of the components of the disclosure comprises 95% of the dosage form for example, capsule volume and/or weight; with a maximum amount of filler (i.e. excipient) being 5%.

Suitable vehicles and excipients are described, for example, in Remington's Pharmaceutical Sciences. The compositions can include dry mixtures of the components formulated in association with one or more pharmaceutically acceptable fillers. The compositions can also include, solutions of the components in association with one or more pharmaceutically acceptable vehicles or diluents, and contained in buffered solutions with a suitable pH and iso-osmotic with the physiological fluids.

Compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween™), alcohols, polyols, glycerin and vegetable oils. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions of the disclosure can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration. For parenteral administration, solutions of a compound described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

Wherein the route of administration is oral, the dosage form may be for example incorporated with carrier or excipient (e.g also referred to as filler) and used in the form of enteric coated tablets, caplets, gelcaps, capsules such as gelatin capsules, ingestible tablets, buccal tablets, troches, elixirs, suspensions, syrups, wafers, and the like.

Suitable excipients include but are not limited to magnesium stearate and/or microcrystalline cellulose.

The excipient can comprise from about 0.1 to about 95% weight/weight of the composition or dosage form. In an embodiment, the excipient comprises about 1%. In another embodiment, the excipient comprises about 2% of the composition. In another embodiment, the excipient comprises about 1% to about 2%, about 3% to about 5%, about 6% to about 10%, about 11% to about 20%, about 21% to about 30%, about 31% to about 40%, about 41% to about 50%, about 51% to about 60%, about 61% to about 70%, about 71% to about 80%, or about 81% to about 90%. In yet a further embodiment, the excipient can comprise up to 3%, up to 5%, up to 10%, up to 15%, up to 20%, up to 30%, up to 40%, up to 50%, up to 60%, up to 70%, up to 80%, up to 80%, up to 90%, or up to 95%.

A further aspect of the disclosure is a composition formulated for as an oral dosage form selected from enteric coated tablets, caplets, gelcaps, and capsules, comprising from about 5 to less than about 2000 mg, suitably from about 10 to about 1000 mg, about 30 to about 600 mg, or about 50 to about 500 mg, of two or more components described herein, and a pharmaceutically acceptable carrier. Suitably each tablet, caplet, gelcap or capsule comprises about 2 to about 2000 mg, suitably about 10 to about 1000 mg, about 100 to about 750 mg, about 200 to about 600 mg or about 350 to about 500 mg of two or more components of the disclosure.

In an embodiment, the dosage form is solid or liquid. For example the solid dosage form can be a powder such as a dry powder that can be reconstituted with for example water, and/or milk. As another example, the liquid dosage form can comprise a beverage product, for example a ready to drink dosage form or a concentrate.

A further aspect of the disclosure is a pharmaceutical composition wherein the dosage form is a solid dosage form comprising from about 2 to about 2000 mg of two or more components of the disclosure, suitably from about 10 to about 1000 mg, about 100 to about 750 mg, about 200 to about 600 mg, or about 350 mg to about 500 mg.

In an embodiment, the solid dosage form is a capsule. In another embodiment, the solid dosage form is suitable for reconstituting into a liquid drink, for example suitable for mixing with water and/or milk.

A further aspect of the disclosure is a pharmaceutical composition wherein the dosage form is a liquid dosage form that contains from about 2 to about 2000 mg of two or more components described herein, suitably from about 10 to about 150 mg, about 10 to about 100 mg, about 20 to about 60 mg, about 35 to about 50 mg, of one or more compounds of the disclosure, in an embodiment two or more components described herein.

Compositions suitable for buccal or sublingual administration include tablets, lozenges, and pastilles, wherein the active ingredient is formulated with a carrier such as sugar, acacia, tragacanth, or gelatin and glycerin.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

The dosage form comprises an effective amount or a therapeutically effective amount. In one embodiment the dosage form comprises about 5 to about 2000 mg of two or more components described herein. In another embodiment, the dosage form comprises about 10 to about 1000 mg of two or more components described herein.

A further aspect of the disclosure is a pharmaceutical composition comprising of two or more components described herein, and/or a pharmaceutically acceptable carrier in unit dosage form in an amount suitable to provide about 0.1 to about 200 mg /kg body weight, suitably about 0.1 to about 100 mg/kg body weight, about 0.1 to about 80 mg/kg body weight, about 0.1 to about 40 mg/kg body weight, about 0.1 to about 20 mg/kg body weight, about 0.1 to about 2 mg/kg body weight or about 0.5 to about 2 mg /kg body weight, formulated into a solid oral dosage form, a liquid dosage form, or an injectable dosage form.

A further aspect of the disclosure is a composition, wherein the amount of two or more components described herein, is an effective amount for the promotion of prostate health and/or the relieving of menstrual related and menopausal related symptoms.

The pharmaceutical compositions are useful for maintaining prostate health, for treating and/or preventing cancer, for example prostate cancer and for treating prostate conditions such as prostate hyperplasia. As well, the compositions are useful for relieving menstrual related and menopausal related symptoms. Accordingly, also included are methods of maintaining prostate health, treating or preventing a cancer or prostate condition, and relieving menstrual related and menopausal related symptoms comprising administering an effective amount of two or more components described herein, to a subject or cell.

Use of such compositions is attractive since no adverse events have been reported for Maca, Epimedium, Cocoa or Quercetin, or rosmarinic acid. All components are currently being sold as natural products in both the US and Canada, and no adverse events have been reported for any of these components to the knowledge of the inventor.

### III. Methods and Uses

### a) Prostate Health

The combination rationale for this product is based on components with multiple modes of action for maintaining healthy prostate function to provide a synergistic effect on the overall reproductive health of the subject.

Epimedium is known to have estrogenic properties, based on the isoprenyl flavonoid icariin and its metabolites. Icariin is known to interact with estrogen receptor beta, and not with the primary alpha receptor. Since estrogens are a traditional form of therapy for prostate cancer, this component at the dosage of for example 125 mg of a 40% icariin Epimedium extract will provide a dose of about 50 mg of phytoestrogen icariin. In preliminary testing, icaritin, a metabolite of icariin was found to inhibit prostate cancer cell growth in vitro and to reduce the growth of human prostatic smooth muscle cells associated with BPH in vitro by inducing apoptosis (Chen et al., 2010). Bone loss is also associated with prostate malfunction (Agrawal et al, 2008) and a clinical trial in women found that Epimedium-derived phytoestrogen flavonoids prevented bone loss in late postmenopausal women (Zhang et al, 2007). Therefore, Epimedium may have secondary benefits.

Red Maca extract reduced ventral prostate size in normal and testosterone enanthate treated rat; Red Maca also prevented the prostate weight increase induced by TE treatment in rats (Gonzales et al., 2005). Maca also contains allyl glucosinolate or sinigrin, which can be converted in the body to isothiocyanates. Isothiocyanates have been shown in animal studies to be an effective cancer fighter. Maca is safe in human trials, does not affect hormone levels, and improves sexual desire. Loss of sexual desire is a known side effect of pharmaceutical treatments for prostatic hyperplasia and prostate cancer. To maintain healthy prostate function, a dose of benzyl glucosinolate in maca will increase sexual desire and may effectively target any cancer cells that may form during the early years of cancer development.

Quercetin, a flavonoid found in onions, is also effective at reducing prostate size. It reportedly has a positive effect on sperm quality and sexual organ function. Most pharmaceutical treatments have negative effects on these areas whereas quercetin has a positive effect. Furthermore, epidemiological studies in New York State demonstrated that increased quercetin intake was correlated with reduced prostate cancer risk (McCann et al., (2005)).

Cocoa polyphenols, have shown both preventative and therapeutic effects in preclinical trials for prostatic hyperplasia and prostate cancer. Cocoa polyphenols both reduce prostate size and weight. In a short term human clinical trial, cocoa polyphenols had a positive effect on blood pressure and vasodilation, and showed no risk factors. Cocoa polyphenols may have secondary health benefits in this combination.

Accordingly, in an embodiment, the disclosure provides a method for maintaining prostate health comprising administering to a subject in need thereof a composition of the disclosure comprising two or more of an icariin component, a quercetin component, a Maca component, and a cocoa polyphenols component. In an embodiment, the composition also comprises a pharmaceutically acceptable carrier or excipient optionally wherein the composition is formulated in unit dosage form. In an embodiment, the composition is suitable for oral administration.

As one or more of the components have been demonstrated to affect prostate cancer cell growth and prostatic hyperplasia cell growth, the composition is in an embodiment, administered for prophylaxis to inhibit prostate cancer or prostatic hyperplasia. In an embodiment, the composition is administered to maintain sexual function.

In a further embodiment, the subject has prostate cancer. In another embodiment, the subject has prostatic hyperplasia. In another embodiment the subject has prostatitis. In a further embodiment, the subject has sexual dysfunction.

Another aspect relates to treating prostate cancer and/or prostatic hyperplasia comprising administering to a subject in need thereof, e.g. a subject with prostate cancer or prostatic hyperplasia, a composition of the disclosure comprising two or more of an icariin component, a quercetin component, a Maca component, and a cocoa polyphenols component.

Another aspect relates to use of a composition comprising two or more of an icariin component, a quercetin component, a Maca component, and a cocoa polyphenols component for maintaining prostate health.

In an embodiment the use is for prophylaxis to inhibit prostate cancer or prostatic hyperplasia. In an embodiment, the use is for prophylaxis to inhibit prostitis. In an embodiment, the use is to maintain sexual function.

A further component provides use of a composition comprising two or more of an icariin component, a quercetin component, a Maca component, and a cocoa polyphenols component for treating prostate cancer, and/or prostatic hyperplasia and/or prostatitis.

In another aspect, the disclosure provides a method of reducing the growth of or inducing cell death of a cancer cell, for example a prostate cancer cell.

In certain embodiments, the cancer cell is in vivo.

In other embodiments, the methods comprise identifying a subject having a cancer cell, prostate cancer, prostatic hyperplasia and/or sexual dysfunction, and administering an effective amount of a composition of the disclosure wherein the composition comprises two or more of the components of the disclosure.

Compositions and the range amounts for each component in the composition suitable for maintaining prostate health, include for example compositions described herein such as compositions comprising from about 5 to about 1000 mg icariin component wherein the icarrin active ingredient is comprised in an Epimedium brevicornum, Epimedium grandiflorum, Epimedium sagittatum extract comprising from about 5% to about 98% icariin active incgredient; about 40 to about 700 mg quercetin active ingredient; about 5 to about 200 mg of cocoa polyphenols active ingredient comprised for example in a Cocoa (Theobroma cacao) extract, the extract comprising from about 5% to about 80% polyphenols; and Maca (Lepidium meyenii) root or hypocotyl extracts which can include for example total Maca root powder; P.E. 4:1; P.E. 6:1;P.E. 10:1 P.E. 15:1; S.E. wherein the extract comprises from about 0.2 to about 5% glucosinolates and/or macamides.

### b) Menstrual Related Symptoms and Menopausal Related Symptoms

Icariin, an isoprenylflavonoid, is considered a phytoestrogen, which targets the beta receptor over the alpha receptor. Clinical studies have also shown that it prevents bone loss in menopausal women, and increases bone density (Zhang et al, 2007). Without wishing to be bound by theory, since estrogen rises at the end of the cycle, the phytoestrogen may interfere with the normal binding to receptors easing pain.

For menstrual pain, one study using an extract of Psidii guajavae folium was used for the management of primary dysmenorrhea. An active ingredient in this extract is quercetin. A double-blinded randomized clinical trial was conducted in 197 women with primary dysmenorrhea. Four intervention groups were used with two extract doses (3 and 6 mg/day) versus ibuprofen (1200 mg/day) and a placebo group at 3 mg/day. Results showed that the group receiving 6 mg/day extract had significantly reduced pain intensity (P<0.001). After discontinuing the treatment, the effect was maintained in cycles 2 and 3, although the reduction in the mean of pain intensity was lower (Doubova, S V; Morales, H R; Hernandez, S F, et al. 2007 J. Ethnopharmacology 110:305-310)

Maca and cocoa have been traditionally used as mood enhancers. Scientific evidence now points to the consumption of cocoa polyphenols and an antidepressant effect (Messaoudi, Michael; Bisson, Jean-Francois; Nejdi, Amine, et al. 2008 Antidepressant-like effects of a cocoa polyphenolic extract in Wistar-Unilever rats. Nutr Neurosci 11:269-276; Haskell, C F; Kennedy, D O; Milne, A L, et al. 2008. Improved cognitive performance and mood in healthy adults following acute consumption of a cocoa flavanol-rich drink. Planta Medica 74:929-929). Maca has reported similar results in the literature in ovariectomized mice (Rubio J; Caldas M; Davila S, et al. 2006. Effect of three different cultivars of Lepidium meyenii (maca) on learning and depression in ovariectomized mice. Complementary and Alternative Medicine 6: June Meeting Abstract 6). The combined effect may promote elevated mood and help relieve symptoms.

Rosmarinic acid is a potent anti-inflammatory (Inoue, K I; Takano, H; Shiga, A, et al. 2005. Effects of volatile constituents of a rosemary extract on allergic airway inflammation related to house dust mite allergen in mice. INTERNATIONAL JOURNAL OF MOLECULAR MEDICINE 16: 315-319). Inflammation involves alterations to vascular and immune cell function. It is well recognized that many physiological reproductive events such as ovulation, menstruation, implantation and onset of labour display hallmark signs of inflammation (Jabbour et al., 2009. Reproduction 138 903-919). As a potent anti-inflammatory, rosmarinic acid would reduce the inflammation associated with menstrual and/or premenstrual symptoms. Rosmarinic acid enters the system within 20 minutes after administration and is converted to 3-O-methyl rosmarinic acid (Baba, S; Osakabe, N; Natsume, M, et al 2005 Absorption, metabolism, degradation and urinary excretion of rosmarinic acid after intake of Perilla frutescens extract in humans EUROPEAN JOURNAL OF NUTRITION 44:1-9). Furthermore, rosmarinic acid has potential preventative effects against AP-1-dependent activation of COX-2 expression.

Several stimuli, including TPA, may activate signaling cascades that lead to the subsequent activation of mitogen activated protein kinase pathways, including the ERK1/2. The ERK1/2pathway contributes to the recruitment of members of the AP-1 factor, which comprises members of the Jun and Fos family of transcription factors, to the COX-2 promoter. Without wishing to be bound by theory, rosmarinic acid may exert preventative effects against the induction of COX-2 transcription by antagonizing the activation of ERK1/2 and the recruitment of AP-1 factors to the CRE harbored in the proximal COX-2 promoter (Scheckel K, Degner S, and D. Romagnolo 2008. Rosmarinic Acid Antagonizes Activator Protein-1-Dependent Activation of Cyclooxygenase-2 Expression in Human Cancer and Non-malignant Cell Lines. The Journal of Nutrition 138:2098-2105.) This anti-inflammatory effect would act synergistically with the other components of the preparation to reduce pain intensity in women suffering from premenstrual syndrome and/or menstrual related symptoms.

Accordingly, an aspect of the disclosure provides a method of relieving menstrual related symptoms comprising administering to a subject a composition of the disclosure, comprising two or more of an icariin component, a quercetin component, a Maca component, a cocoa polyphenols component and a rosmarinic acid component.

Another aspect provides a method of treating menstrual related symptoms comprising administering to a subject a composition of the disclosure, comprising two or more of an icariin component, a quercetin component, a Maca component, a cocoa polyphenols component and a rosmarinic acid component.

In an aspect, the disclosure provides a method of relieving premenstrual syndrome comprising administering to a subject a composition of the disclosure, comprising two or more of an icariin component, a quercetin component, a Maca component, a cocoa polyphenols component and a rosmarinic acid component.

Another aspect provides a method of treating premenstrual syndrome comprising administering to a subject a composition of the disclosure, comprising two or more of an icariin component, a quercetin component, a Maca component, a cocoa polyphenols component and a rosmarinic acid component.

A further aspect relates to a method of relieving menopausal related symptoms comprising administering to a subject a composition of the disclosure, comprising two or more of an icariin component, a quercetin component, a Maca component, a cocoa polyphenols component and a rosmarinic acid component.

Yet a further aspect relates to a method of treating menopausal related symptoms comprising administering to a subject a composition of the disclosure, comprising two or more of an icariin component, a quercetin component, a Maca component, a cocoa polyphenols component and a rosmarinic acid component.

Another aspect provides use of a composition of the disclosure comprising two or more of an icariin component, a quercetin component, a Maca component, a cocoa polyphenols component and a rosmarinic acid component for relieving menstrual related symptoms, such as premenstrual syndrome or menopausal related symptoms.

A further aspect provides use of a composition of the disclosure comprising two or more of an icariin component, a quercetin component, a Maca component, a cocoa polyphenols component and a rosmarinic acid component for treating menstrual related symptoms, such as premenstrual syndrome or menopausal related symptoms.

Compositions and the range amounts for each component in the composition suitable for relieving or treating menstrual related symptoms, including premenstrual syndrome and menopausal related symptoms include for example compositions disclosed herein such as compositions comprising from about 5 to about 1000 mg icariin component wherein the icariin active ingredient is comprised for example in a Epimedium brevicornum, Epimedium grandiflorum, Epimedium sagittatum extract, for example, comprising from about 5% to about 98% icariin active ingredient; about 40 to about 700 mg quercetin active ingredient; about 5 to about 200 mg of cocoa polyphenols active ingredient comprised for example in a Cocoa (Theobroma cacao) extract, the extract comprising from about 5% to about 80% polyphenols; Maca (Lepidium meyenii) root or hypocotyl extracts which can include for example total Maca root powder; P.E. 4:1; P.E. 6:1;P.E. 10:1 P.E. 15:1; S.E. wherein the extract comprises from about 0.2 to about 5.0% glucosinolates and/or macamides; and about 20 to about 500 mg of rosmarinic acid active ingredient wherein the rosmarinic acid is comprised for example in an extract of Rosmarinus officinalis, Mentha spicata, Origanum sp, Thymus sp. Prunella sp. Melissa, Salvia, Perilla and/or other herb comprising rosmarinic acid, wherein the extract comprises from about 2 to about 80% rosmarinic acid active ingredient.

Any quantity between the disclosed ranges described herein is also contemplated.

### Dosages for Methods/Uses

The composition is administered for example at an effective amount to reduce or inhibit at least one symptom or negative change in the health of the prostate, or at least one symptom associated with menstruation, premenstrual syndrome or menopause.

In an embodiment, the recommended dosage is about 1 to about 3 or more unit dosage forms such as capsules per day. In an embodiment, the recommended dosage is about 1, or 1 unit dosage form, e.g. capsule per day. In another embodiment, the recommended dosage is about 2, or 2 unit dosage forms e.g. capsules per day. In another embodiment, the recommended dose is about 3, or 3 unit dosage forms e.g. capsules per day. In yet a further embodiment, the recommended dose is up to 3 unit dosage forms e.g. capsules per day.

In another embodiment, the recommended dosage is about 1 to about 3 unit dosage forms about every 4 hours, about every 6 hours or about every 8 hours as needed. In another embodiment, the recommended dosage is 1 unit dosage forms about every 4 hours as needed. For example, during periods of discomfort and/or pain, such as relating from menstrual related symptoms, a subject may //takel capsule every 4 hours as needed for a maximum of about 6 unit dosage forms per day.

In another embodiment, the recommended dosage is taken daily. In another embodiment, the recommended dosage is taken daily, for up to 1, 2, 3, 4, or more weeks. In a further embodiment, the recommended dosage is taken daily for up to 1, 2, 3, 4, 5, 6, 7, 8, 9, 10, 11 or 12 months.

In an embodiment, the unit dosage form, optionally a capsule, comprises a composition of the disclosure.

In an additional aspect of the disclosure, the effective amount, is within the range of about 0.1 to about 200 mg/kg body weight, suitably in the range of about 0.1 to about 100 mg/kg body weight, about 0.1 to about 80 mg/kg body weight, about 0.1 to about 40 mg/kg body weight, about 0.1 to about 20 mg/kg body weight, about 0.1 to about 2 mg/kg body weight, or about 0.5 to about 2 mg/kg body weight. In another embodiment, the effective amount is within the range of about 5 to about 50 mg/kg body weight, about 10 to about 50 mg/kg body weight, about 20 to about 50 mg/kg body weight, about 5 to about 40 mg/kg body weight, or about 10 to about 40 mg/kg body weight.

In other embodiments, the effective amount is within the range of about about 2 mg to about 4000 mg, about 50 mg to about 4000 mg, about 100 mg to about 4000 mg, about 250 mg to about 4000 mg, about 500 mg to about 4000 mg, about 1000 mg to about 4000 mg, about 2000 mg to about 4000 mg, about 2 mg to about 2000 mg, about 10 mg to about 2000 mg, about 100 mg to about 2000 mg, about 250 mg to about 2000 mg, about 500 mg to about 2000 mg, about 1000 mg to about 2000 mg, about 10 mg to about 1000 mg, about 100 mg to about 1000 mg, about 250 mg to about 1000 mg, about 500 mg to about 1000 mg, about 50 mg to about 500 mg, about 100 mg to about 500 mg, about 10 mg to about 150 mg, about 10 mg to about 100 mg, about 20 mg to about 60 mg, or about 35 mg to about 50 mg optionally daily dosage and administration or use is one or more times daily for about 1 to about 2 days, about 1 to about 2 weeks, about 2 to about 4 weeks, and/or more than about 4 weeks.

Also provided are methods and uses wherein the compound is comprised in a dosage form described herein.

A further aspect of the disclosure is a commercial package comprising a composition according to the present disclosure, and associated therewith instructions for the use thereof for example for maintaining prostate health or relieving menopausal related symptoms.

The following non-limiting examples are illustrative of the present disclosure:

### EXAMPLES

### Example 1

Men's Prostate Health Formulation

The men's prostate health formulation consists of 165 mg of a 40% icariin extract of Epimedium sagittatum (Barrenwort) powder, 135 mg of a 10:1 purified extract of Lepidium meyenii (Maca) root powder, 95 mg of a 40% Cocoa Activ™ cocoa polyphenol extract from Theobroma cocoa (chocolate beans), 65 mg of 95% pure quercetin flavonoid and 1-2% magnesium stearate as a filler, all encapsulated in a gelatin capsule. One capsule is to be taken daily. Manufacturing of the Men's Prostate Health Formula was performed at Promising Health, Markham Ontario. All Bacterial, heavy metal, pesticides and solvent tests were performed (lab chemical tests were performed by Labs-Mart Inc. 9415 20th Ave NW Edmonton, AB) and conformed to Health Canada standards.

Cocoa Activ™ is a trademark of Naturex Inc.,South Hackensack N.J., USA.

### Example 2

Women's Peri-Postmenopausal Health

The women's Peri-Postmenopausal health formulation consists of 165 mg of a 40% icariin extract of Epimedium sagittatum (Barrenwort) powder (Stryka Botanics, Hillsborough, N.J., USA), 100 mg of a 10:1 purified extract of Lepidium meyenii (Maca) root powder (Stryka Botanics, Hillsborough, N.J., USA), 95 mg of a 40% Cocoa Activ™ cocoa polyphenol extract from Theobroma cocoa (chocolate beans) (Naturex, South Hackensack N.J., USA), 100 mg of 95% pure quercetin flavonoid (Stryka Botanics, Hillsborough, N.J., USA) 100 mg of 40% rosmarinic acid extract of Rosmarinus officinalis (Rosemary) (PL Thomas Morristown, N.J., USA) and 1-2% magnesium stearate as a filler, all encapsulated in a gelatin capsule. One capsule is to be taken daily.

### Example 3

Women's Premenstrual Health

The women's Premenstrual health formulation consists of 100 mg of a 10:1 purified extract of Lepidium meyenii (Maca) root powder (Stryka Botanics, Hillsborough, N.J., USA), 100 mg of a 40% Cocoa Activ™ cocoa polyphenol extract from Theobroma cocoa (chocolate beans) (Naturex, South Hackensack N.J., USA), 100 mg of 95% pure quercetin flavonoid (Stryka Botanics, Hillsborough, N.J., USA), 300 mg of 40% rosmarinic acid extract of Rosmarinus officinalis (Rosemary) (PL Thomas Morristown, N.J., USA) and 1-2% magnesium stearate as a filler, all encapsulated in a gelatin capsule. One capsule can be taken every 4 hours.

### Example 4

Preliminary Testing for Changes in Human Subjects

Method: Volunteers were provided with a 30 day supply of the combination at 450 mg per day by taking one capsule per day. The volunteers where asked to fill in a questionnaire designed to assess the mood, energy, urine flow, sleep patterns, and weekly monitoring of blood pressure. Results: Preliminary test on 6 volunteers (4 men ages 25-45, 2 women ages 35 and 47) each reporting the energy spike after 3-5 days after dosing at 1 capsule per day. The energy level was maintained for the duration of the study. No changes in sleep patterns, level of appetite, stress, mood, body weight were recorded. Exercise tolerance and mental concentration were recorded as slightly increased. Men reported that they experienced increased urinary flow.

The two women reported a mood improvement during premenstrual period and an anti-inflammatory effect (relief of menstrual pain). No changes in sleep patterns were recorded.

### Example 5

Method: A dose dependent study will be performed on PC-3 prostate cancer cell lines were purchased from American Type Culture Collection (ATCC, Manassa, Va.) and will be grown in a standard method in 75 cm² flasks in RPMI-1640 medium, 37° C. and 5% CO₂. For the growth assays cells will be plated in 96-well plates and then allowed to attach overnight in 10% fetal bovine serum (FBS). Growth will be determined after 48 h by the CellTiter 96AQ assay (Promega, Madison, Wis.) to establish a baseline rate. For apoptosis of the PC3 cells, a predetermined level of cells per well will be plated in the 96-well plates and treated as in the growth assays. Dose dependent levels of the combination as well as the separate components will be added to half of the wells and compared to a placebo control. Apoptosis will be determined by Cell Death Detection ELISA Plus (Roche, Applied Science, Indianapolis, Ind.). Results should indicate that there is a synergistic effect of all components compared to each component used separately.

### Example 6

Method: A comparative study to determine the synergistic effect of the combination on induced prostatic hyperplasia. Rats will be treated with testosterone enanthate to induce prostatic hyperplasia and then subjected to three different treatments; 0.1 mg of finasteride which is the accepted pharmaceutical treatment for prostatic hyperplasia; 0.1 mg benzylglucosinolate, which has been shown in the literature to be as effective as finasteride, and an equivalent dose of the combination. Prostate weights of the treated groups will be compared to a control group to determine whether the treatments reduce prostate weight in rats with prostatic hyperplasia and whether the combination shows a synergistic effect compared to the benzylglucosinolate alone.

### Example 7

Method: A double blind cross-over placebo study will be performed on a group of 20 men aged 40-60 with mildly elevated Prostate Specific Antigen (PSA) levels to determine the effect of the combination on lower PSA levels after a 30 day treatment. Patients will be tested for both Free and total PSA levels prior to initiating treatment, and given either a placebo or the combination therapy. After 30 days, the patients will be tested for their dual PSA levels and then subjected to a 14 day washout period. Subsequently, patients will be then subjected to the opposite treatment for another 30 days and retested for their dual PSA levels. The study will be run in conjunction with the Human Nutraceutical Research Unit at the University of Guelph. The expected outcome is to reduce PSA levels after the combination therapy.

### Example 8

Method: A placebo controlled study will be performed on a group of 20 females to examine the effect of the combination on relieving menstrual pain and modifying mood during premenstruation. The study will consist of a 60 day treatment in which participants will answer a detailed questionnaire regarding their inflammatory pain, symptoms, sleep patterns, energy levels, and moods over the 60 day treatment program. The answers will be numerically rated such that statistics will be performed on the numbers so that the effects of the combination can be compared with the effects of the placebo control.

### Example 9

Method: A placebo controlled study will be performed on a group of 30 females to examine the effect of the combination on modifying mood and other symptomatic factors on postmenopausal women. The study will consist of a 60 day treatment in which participants will answer a detailed questionnaire regarding their sleep patterns, energy levels, and mood swings over the 60 day treatment program. The answers will be numerically rated such that statistics will be performed on the numerical values generated by the questionnaire so that the effects of the combination can be compared with that of the placebo control.

### Example 10

A men's prostate health formulation consists of 123 mg of a 40% icariin extract of Epimedium sagittatum (Barrenwort) powder (Stryka Botanics, Hillsborough, N.J., USA), 172 mg of a 10:1 purified extract (P.E.) of Lepidium meyenii (Maca) root powder (Stryka Botanics, Hillsborough, N.J., USA), 93 mg of a 40% Cocoa Activ™ cocoa polyphenol extract from Theobroma cocoa (chocolate beans) (Naturex, South Hackensack N.J., USA), 62 mg of 95% pure quercetin flavonoid (Stryka Botanics, Hillsborough, N.J., USA) and 1-2% magnesium stearate as a filler, all encapsulated in a gelatin capsule. One capsule is to be taken daily for men under 100 kg and 2 capsules can be taken daily for men over 100 kg.

Manufacturing of the Men's Prostate Health Formula was performed at Promising Health, Markham Ontario. All Bacterial, heavy metal, pesticides and solvent tests were performed (lab chemical tests were performed by Labs-Mart Inc. 9415 20th Ave NW Edmonton, AB) and microbial tests by Alpharmco, 3400 Blvd Losh #24 St. Hubert QC) and conformed to Health Canada standards.

Cocoa Activ™ is a trademark of Naturex Inc., South Hackensack N.J., USA.

### Example 11

Women's Peri-Postmenopausal Health

A women's Peri-Postmenopausal health formulation consists of 100 mg of a 40% icariin extract of Epimedium sagittatum (Barrenwort) powder (Stryka Botanics, Hillsborough, N.J., USA), 100 mg of a 10:1 purified extract of Lepidium meyenii (Maca) root powder (Stryka Botanics, Hillsborough, N.J., USA), 100 mg of a 40% Cocoa Activ™ cocoa polyphenol extract from Theobroma cocoa (chocolate beans) (Naturex, South Hackensack N.J., USA), 100 mg of 95% pure quercetin flavonoid (Stryka Botanics, Hillsborough, N.J., USA) 100 mg of 40% rosmarinic acid extract of Rosmarinus officinalis (Rosemary) (PL Thomas Morristown, N.J., USA) and 1-2% magnesium stearate as a filler, all encapsulated in a gelatin capsule. One capsule is to be taken daily.

### Example 12

Women's Premenstrual Health

A women's Premenstrual health formulation consists of 75 mg of a 10:1 purified extract of Lepidium meyenii (Maca) root powder (Stryka Botanics, Hillsborough, N.J., USA), 100 mg of a 40% Cocoa Activ™ cocoa polyphenol extract from Theobroma cocoa (chocolate beans) (Naturex, South Hackensack N.J., USA), 100 mg of 95% pure quercetin flavonoid (Stryka Botanics, Hillsborough, N.J., USA), 300 mg of 40% rosmarinic acid extract of Rosmarinus officinalis (Rosemary) (PL Thomas Morristown, N.J., USA) and 1-2% magnesium stearate as a filler, all encapsulated in a gelatin capsule. One capsule can be taken every 4 hours. The reduction in the amount of the Maca is due to the frequency of dosage, and increased amounts of rosmarinic acid are required to maintain anti-inflammatory activity associated with menstruation.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### CITATIONS FOR REFERENCES REFERRED TO IN THE SPECIFICATION

Agrawal, M M, Rana S, Mandal A K, Malhotra S, Khandelwal N, Kumar S, et al. (2008) Lactose intolerance in prostate cancer patients: Incidence and associated factors. Scandinavian Journal of Gastroenterology 43: 270-276.
Baba, S; Osakabe, N; Natsume, M, et al (2005) Absorption, metabolism, degradation and urinary excretion of rosmarinic acid after intake of Perilla frutescens extract in humans EUROPEAN JOURNAL OF NUTRITION 44:1-9.
Bisson J F, Hidalgo S, Rozan P, and M Messaoudi. (2007) Preventive effects of ACTICOA powder, a cocoa polyphenolic extract on experimentally induced prostate hyperplasia in Wistar-Unilever rats. Journal of Medicinal Food 10:622-627.
Bisson J F, Hidalgo S, Rozan P, and M Messaoudi. (2007) Therapeutic effect of ACTICOA powder, a cocoa polyphenolic extract, on experimentally induced prostatic hyperplasia in Wistar-Unilever rats. Journal of Medicinal Food 10: 628-635.
Bisson J F, Guardia-Llorens M A, Hidalgo S, Rozan P, and M Messaoudi. (2008) Protective effect of Acticoa powder, a cocoa polyphenolic extract, on prostate carcinogenesis in Wistar-Unilever rats. European Journal of Cancer Prevention 17:54-61.
Chen M F, Qi L, Li Y, Zu X-B, Yuan-Qin Dai Y-Q, and P Zhang (2010) Icaritin induces growth inhibition and apoptosis of human prostatic smooth muscle cells in an estrogen receptor-independent manner. Amino Acids 38:1505-1513.
Circero, A F G. (2010) Drug Plants, in Recent Progress in Medicinal Plants 27:347-362.
Doubova, S V; Morales, H R; Hernandez, S F, et al. (2007) Effect of a Psidii guajavae folium extract in the treatment of primary dysmenorrhea: A randomized clinical trial. J. Ethnopharmacology 110:305-310.
Garcia-Ca~nas V, Simó C León C, and A. Cifuentes Advances in Nutrigenomics research: Novel and future analytical approaches to investigate the biological activity of natural compounds and food functions. Journal of Pharmaceutical and Biomedical Analysis 51 (2010) 290-304.
Gonzales G F, Cordova A, Vega K, Chung A, Villena A, Gonez C, and S Castillo. (2002) Effect of Lepidium meyenii (MACA) on sexual desire and its absent relationship with serum testosterone levels in adult healthy men. Andrologia 34: 367-372.
Gonzales G F, Cordova A, Vega K, Chung A, Villena A, and C Gonez. (2003) Effect of Lepidium meyenii (Maca), a root with aphrodisiac and fertility enhancing properties, on serum reproductive hormone levels in adult healthy men. Journal of Endocrinology 176: 163-168.
Gonzales G F, Miranda S, Nieto J, Fernandez G, Yucra S, Rubio J, Pedro Y, and M Gasco. (2005) Red maca (Lepidium meyenii) reduced prostate size in rats. Reproductive Biology and Endocrinology 3: 5-20.
Gonzales G F, Vasquez V, Rodriguez D, Maldonado C, Mormontoy J, Portella J, Pajeulo M, Villegas L, and M Gasco. (2007) Effect of two different extracts of red maca in male rats with testosterone induced prostatic hyperplasia. Asian Journal of Andrology 9: 245-251.
Gonzales G F, Gasco M, Malheiros-Pereira A, and C. Gonzales-Castaneda.

(2008) Antagonistic effect of Lepidium meyenii (red maca) on prostatic hyperplasia in adult mice. ANDROLOGIA 40 :179-185.
Gong Y, Yip S C, Tharmarai S K, Zhang J, Lee H K, and E L Yong. (2007) Trace analysis of icariin in human serum with dansyl chloride derivatization after oral administration of Epimedium decoction by liquid chromatography tandem mass spectrometry. Journal of Chromatography B 860: 166-172.
Haskell, C F; Kennedy, D O; Milne, A L, et al. (2008). Improved cognitive performance and mood in healthy adults following acute consumption of a cocoa flavanol-rich drink. Planta Medica 74:929-929
Huang X, Zhu D, and Y Lou. (2007) A novel anticancer agent, icaritin, induced cell growth inhibition, G1 arrest, and mitochondrial transmembrane potential drop in human prostate carcinoma PC-3 cells. European Journal of Pharmacology 564: 26-36.
Inoue, K I; Takano, H; Shiga, A, et al. (2005) Effects of volatile constituents of a rosemary extract on allergic airway inflammation related to house dust mite allergen in mice. INTERNATIONAL JOURNAL OF MOLECULAR MEDICINE 16: 315-319.
Jabbour H, Sales K, Catalano R, and J Norman (2009). Inflammatory pathways in female reproductive health and disease. Reproduction 138 903-919
McCann S E, Ambrosone C B, Moysich K B, Brasure J, Marshall J R, Freudenheim J L et al. (2005) Intakes of selected nutrients foods and phytochemicals and prostate cancer risk in western New York. Nutrition and Cancer 53: 33-41.

McCollom, M M, Villinski J R, McPhail, K L, Craker, L E, Gafner, S. (2005) Analysis of macamides in samples of Maca (Lepidium meyenii) by HPLC-UV-MS/MS. Phytochemical Anal 16:463-469).
Messaoudi, Michael; Bisson, Jean-Francois; Nejdi, Amine, et al. (2008) Antidepressant-like effects of a cocoa polyphenolic extract in Wistar-Unilever rats. Nutr. Neurosci 11:269-276.
Moon Y J, Wang L, DiCenze R, and ME Morris. (2008) Quercetin pharmacokinetics in humans. Biopharmaceutics and Drug Disposition 29: 205-217.
Rubio J; Caldas M; Davila S, et al. (2006). Effect of three different cultivars of Lepidium meyenii (maca) on learning and depression in ovariectomized mice. Complementary and Alternative Medicine 6: June Meeting Abstract 6.
Scheckel K, Degner S, and D. Romagnolo (2010). Rosmarinic Acid Antagonizes Activator Protein-1-Dependent Activation of Cyclooxygenase-2 Expression in Human Cancer. The Journal of Nutrition 138:2098-2105.
Shen P, Wong S P, and E L Yong. (2007) Sensitive and rapid method to quantify icaritin and desmethylicaritin in human serum using gas chromatography-mass spectrometry. Journal of Chromatography B 857: 47-52.
Shen P, Guo B L, Gong Y, Hong D Y, Hong Y, and E L Yong. (2007) Taxonomic, genetic, chemical and estrogenic characteristics of Epimedium species. Phytochemistry 68:1448-1458.
Taepongsorat L, Tangpraprutgul P, Kitana N and S Malaivijitnond. (2008) Stimulating effects of quercetin on sperm quality and reproductive organs in adult male rats. Asian Journal of Andrology 10: 249-258.
Taubert D, Roesen R, Lehmann C, Jung N, and E Schomig. (2007) Effects of low habitual cocoa intake on blood pressure and bioactive nitric oxide. A randomized controlled trial. Journal of the American Medical Association 298: 49-60.
Vijayababu M R, Arunkumar A, Kanagaraj P, Venkataranman P, Krishnamoorthy G, and J Arunkaran. (2006) Quercetin downregulates matrix metalloproteinases 2 and 9 proteins expression in prostate cancer cells (PC-3). Molecular and Cellular Biochemistry 287: 109-116.
Zhang G, Qin L, and Y Shi. (2007) Epimedium derived phytoestrogen flavonoids exert beneficial effect on preventing bone loss in late postmenopausal women: a 24 month randomized, double-blind and placebo-controlled trial. Journal of Bone and Mineral Research 22: 1072-1079.

We claim:

1. A composition comprising two or more of:
a. an icariin component;
b. a Maca component;
c. a quercetin component;
d. a cocoa polyphenols component; and
e. a rosmarinic acid component.

2. The composition of claim 1 wherein the icariin component comprises at least 5% to at least 98% of an icariin active ingredient and/or the amount of the icariin active ingredient in the composition is at least or about 5 mg to least or about 1000 mg.

3. The composition of claim 1, wherein the icariin component comprises an extract, optionally an extract of an Epimedium species, Vancouveria hexandra, and/or Ranzania japonica, optionally wherein the Epimedium species is selected from Epimedium brevicornum, Epimedium grandiflorum and Epimedium sagittatum.

4. The composition of claim 1, wherein the quercetin component comprises at least 5% to at least 98% of a quercetin active ingredient and/or the amount of the quercetin active ingredient in the composition is at least or about 20 mg to at least or about 700 mg.

5. The composition of claim 1, wherein the cocoa polyphenols component comprises at least or about 5% to at least or about 90% of a cocoa polyphenols active ingredient and/or the amount of the cocoa polyphenols active ingredient in the composition is at least or about 5 mg to at least or about 200 mg.

6. The composition of claim 5 wherein the cocoa polyphenols component comprises an extract optionally an extract of Theobroma cacao.

7. The composition of claim 1, wherein the Maca component comprises at least or about 0.2% to at least or about 10% of glucosinolates and/or macamides, optionally Maca root powder with a P.E. 4:1, P.E. 6:1, P.E. 10:1, P.E. 15:1 or a S.E of about 0.2% to about 10% glucosinolates and/or macamides, and wherein the amount of the Maca component in the composition optionally is at least or about 15 mg to at least or about 1000 mg.

8. The composition of claim 1, wherein the Maca component comprises an extract of total Maca root or Maca hypocotyls, and/or a combination thereof.

9. The composition of claim 7 wherein the glucosonilates comprises benzylglucosinolate and/or allylglucosinolate, optionally wherein the amount of benzyl glucosinolate in the composition is within the range of 0.1 mg to about 15 mg.

10. The composition of claim 1, wherein the rosmarinic acid component comprises at least or about 2% to at least 90% of a rosmarinic acid active ingredient, and wherein the amount of the rosmarinic acid active ingredient is optionally at least or about 20 mg to at least or about 500 mg.

11. The composition of claim 10, wherein the rosmarinic component comprises an extract, optionally an extract of Rosmarinus officinalis, Mentha spicata, Origanum sp, Thymus sp. Prunella sp. Melissa, Salvia, Lamiaceae herb and/or Perilla and/or combinations thereof.

12. The composition of claim 1, wherein the composition is a pharmaceutical composition, optionally wherein the pharmaceutical composition is formulated as an oral dosage form.

13. The composition of claim 1, wherein the composition is formulated for use as a liquid drink such as a tea.

14. The composition of claim 1 further comprising an excipient, optionally wherein the excipient is magnesium stearate.

15. The composition of claim 12, wherein the oral dosage form is selected from enteric coated tablets, caplets, gelcaps, and capsules and comprises from about 5 mg to less than about 2000 mg, from about 10 mg to about 1000 mg, about 30 mg to about 600 mg, or about 50 mg to about 500 mg, of two or more of the icariin component, the Maca component, the quercetin component, the cocoa polyphenols component and the rosmarinic acid component, and a pharmaceutically acceptable carrier.

16. A method for maintaining prostate health, and/or treating prostate cancer and/or prostatic hyperplasia comprising administering to a subject in need thereof a therapeutically effective amount of the composition of claim 1.

17. The method of claim 16, wherein the composition is administered for prophylaxis to inhibit prostate cancer or prostatic hyperplasia and/or to maintain sexual function.

18. A method of relieving and/or treating premenstrual, menstrual related and/or menopausal symptoms comprising administering to a subject in need thereof a therapeutically effective amount of the composition of claim 1.

19. The method of claim 16 wherein the subject is administered a recommended dosage, optionally, wherein the recommended dosage is about 1 to about 3 or more capsules per day; is at least 1 or 1 capsule per day; or is about 1 to about 3 capsules about every 4, about every 6, or about every 8 hours as needed.

20. The method of claim 18, wherein the subject is administered a recommended dosage, optionally wherein the recommended dosage is about 1 to about 3 or more capsules per day; is at least 1 or 1 capsule per day; or 1 to about 3 capsules about every 4, about every 6, or about every 8 hours as needed.
